SEQUENCE LISTING (14 sequences)

SEQ ID NO: 1 - protein, 939 aa, Artificial Sequence
  Synthetic
MASMQKLISEEDLLMAMEARIRSTVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRQVAHSGAKASVVDGTPLVAAPSLNATTVVTTVYQEPIMSQGAALSGEPTTLTKEEEKKQPDEEPMDMVVEKQEETDHKNDNQILSEIVEAKMAEELKPMDTDKESIAESKSPEMSMQEDCISDIAPMQTDEQTNKEQFVPGPNEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPPLTPTSKVYTIRPYFPKDEASVYKICREMYDDGVGLPFQSQPDLIGDKLVGGLLSLSLDYCFVLEDEDGICGYALGTVDVTPFIKKCKISWIPFMQEKYTKPNGDKELSEAEKIMLSFHEEQEVLPETFLANFPSLIKMDIHKKVTDPSVAKSMMACLLSSLKANGSRGAFCEVRPDDKRILEFYSKLGCFEIAKMEGFPKDVVILGRSL

SEQ ID NO: 2 - DNA, 2820 bp, Artificial Sequence
  Synthetic
atggcatcaatgcagaagctgatctcagaggaggacctgcttatggccatggaggcccgaattcggtcgaccgtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggcaagttgcacacagtggagctaaagcaagtgtagttgatgggactcctttagttgcagcaccctctttaaatgccacaaccgtagtaacaacagtttatcaggagcccattatgagccagggagcagccttgagtggtgagcctactactctgaccaaggaagaagaaaagaaacagcctgatgaagaacccatggacatggtggtggaaaaacaagaagaaacggaccacaagaatgacaatcaaatactgagtgaaattgttgaagcgaaaatggcagaggaattgaaaccaatggacactgataaagagagcatagctgaatcaaaatccccagagatgtccatgcaagaagattgtattagtgacattgcccccatgcaaactgatgaacagacaaacaaggagcagtttgtgccaggtccaaatgaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccacctccactgactcctacctccaaagtttatactatcagaccttattttcctaaggatgaggcatccgtgtacaagatttgcagagaaatgtatgacgatggagtgggtttaccctttcaaagtcagcctgatcttattggagacaagttagtaggagggctgctttccctcagcctggattactgctttgtcctagaagatgaagatggcatatgtggttatgccttgggcactgtagatgtgaccccctttattaaaaaatgtaaaatttcctggatccccttcatgcaggagaagtataccaagccaaatggtgacaaggaactctctgaggctgagaaaataatgttgagtttccatgaagaacaggaagtactgccagaaactttccttgctaatttcccttctctgataaagatggacattcacaaaaaagtaactgacccaagtgtggccaaaagcatgatggcttgcctcctgtcttcactgaaggctaatggctcccggggagctttctgtgaagtgagaccagatgataaaagaattctggaattttacagcaagttaggatgttttgaaattgcaaaaatggaaggatttccaaaggatgtggttatacttggtcggagcctgtaa

SEQ ID NO: 3 - DNA, 1245 bp, Artificial Sequence
  Synthetic
atggagcagaagctgatcagcgaggaggacctggcgatcgcaatggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggtaa

SEQ ID NO: 4 - protein, 414 aa, Artificial Sequence
  Synthetic
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSR

SEQ ID NO: 5 - DNA, 1245 bp, Artificial Sequence
  Synthetic
atggagcagaagctgatcagcgaggaggacctggcgatcgcaatggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttaatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggtaa

SEQ ID NO: 6 - protein, 414 aa, Artificial Sequence
  Synthetic
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFNDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSR

SEQ ID NO: 7 - DNA, 534 bp, Artificial Sequence
  Synthetic
atggcatacccatacgatgttccagattacgctgcgatcgcagaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccaccttaa

SEQ ID NO: 8 - protein, 177 aa, Artificial Sequence
  Synthetic
MAYPYDVPDYAAIAEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 9 - DNA, 957 bp, Artificial Sequence
  Synthetic
atggcatacccatacgatgttccagattacgctgcgatcgcacaggtgcagctggtggagtctggaggagctctggtgcagcctggaggaagcctgcgcctgagctgtgcagctagcggatttcctgtgaaccgctacagcatgcgctggtaccgccaggctcctggtaaagagcgcgagtgggtggctggaatgagcagcgctggagatcgcagcagctacgaggacagcgtgaaaggacgctttacaatcagccgcgatgatgctcgcaacacagtgtacctgcagatgaactctctgaaacctgaggacactgctgtgtactactgtaacgtgaacgtgggtttcgagtactggggacagggaacacaggtgacagtgagctctggcgcgccagaggcagctgcaaaggaggcagctgcaaaggaggcagctgcaaaggaggcagctgcaaagttaattaaggaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctaaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccaccttaa

SEQ ID NO: 10 - protein, 318 aa, Artificial Sequence
  Synthetic
MAYPYDVPDYAAIAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPEAAAKEAAAKEAAAKEAAAKLIKEKPLYTAEPVTLEDLQLLADLFYLPYEHGPKGAQMLREFQWLRANSSVVSVNCKGKDSKKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 11 - protein, 14 aa, Artificial Sequence
  Synthetic
QADQEAKELARQIA

SEQ ID NO: 12 - protein, 15 aa, Artificial Sequence
  Synthetic
GSGSGSGSGSGSGSG

SEQ ID NO: 13 - protein, 12 aa, Artificial Sequence
  Synthetic
PDRKAAVSHWQQ

SEQ ID NO: 14 - protein, 8 aa, Artificial Sequence
  Synthetic
DYKDDDDK

(19) United States
(12) Patent Application Publication
(10) Pub. No.: US 2024/0043528 A1
(43) Pub. Date: Feb. 8, 2024

(54) NANOBODY-OGA FUSIONS AND USES THEREOF

(71) Applicant: President and Fellows of Harvard College, Cambridge, MA (US)

(72) Inventors: Christina M. Woo, Cambridge, MA (US); Yun Ge, Cambridge, MA (US); Daniel Hector Ramirez, Cambridge, MA (US)

(73) Assignee: President and Fellows of Harvard College, Cambridge, MA (US)

(21) Appl. No.: 18/030,245

(22) PCT Filed: Oct. 4, 2021
(86) PCT No.: PCT/US2021/053421

§ 371 Date: Apr. 4, 2023

Related U.S. Application Data
(60) Provisional application No. 63/158,244, filed on Mar. 8, 2021.
(60) Provisional application No. 63/087,773, filed on Oct. 5, 2020.

(51) Int. Cl. C07K 16/28; C12N 15/63; C12N 9/24

(52) U.S. Cl. CPC C07K 16/28; C12N 15/63; C12N 9/2402; C12Y 302/01169; C07K 2317/569; C07K 2319/00; C07K 2317/41

(57) ABSTRACT

The present disclosure provides fusion proteins comprising a nanobody and a split glycosyl hydrolase enzyme. Also provided herein are split glycosyl hydrolase enzymes and fusion proteins comprising such enzymes. Further provided herein are polynucleotides, vectors, and cells. The present disclosure also provides methods of deglycosylating a protein and methods of studying the effects of glycosylation on protein function in cells. Also provided herein are methods of treating diseases.

### RELATED APPLICATIONS

This application claims priority under 35 U.S.C. § 119(e) to U.S. Provisional Application U.S. Ser. No. 63/158,244, filed Mar. 8, 2021, and to U.S. Provisional Application U.S. Ser. No. 63/087,773, filed Oct. 5, 2020, the contents of each of which are incorporated herein by reference.

### BACKGROUND OF INVENTION

O-Linked N-acetyl glucosamine (O-GlcNAc) is a monosaccharide post-translational modification (PTM) installed on serine or threonine residues of numerous nucleocytoplasmic proteins across species (Yang, X. & Qian, K. Protein O-GlcNAcylation: emerging mechanisms and functions. Nat. Rev. Mol. Cell Biol. 2017, 18, 452-465). O-GlcNAc is a reversible and dynamic modification regulated by a single pair of enzymes, the writer O-GlcNAc transferase (OGT) (Levine, Z. G. & Walker, S. The Biochemistry of O-GlcNAc Transferase: Which Functions Make It Essential in Mammalian Cells? Annu. Rev. Biochem. 2016, 85, 631-57) and the eraser O-GlcNAcase (OGA) (Alonso, J., Schimpl, M. & van Aalten, D. M. O-GlcNAcase: promiscuous hexosaminidase or key regulator of O-GlcNAc signaling? J. Biol. Chem. 2014, 289, 34433-9). Loss of O-GlcNAc homeostasis has been linked to many diseases, including neurodegeneration (Yuzwa, S. A. & Vocadlo, D. J. O-GlcNAc and neurodegeneration: biochemical mechanisms and potential roles in Alzheimer's disease and beyond. Chem Soc Rev 2014, 43, 6839-58), diabetes (Ma, J. & Hart, G. W. Protein O-GlcNAcylation in diabetes and diabetic complications. Expert Rev Proteomics 2013, 10, 365-80), and cancer (Slawson, C. & Hart, G. W. O-GlcNAc signalling: implications for cancer cell biology. Nat. Rev. Cancer 2011, 11, 678-84). Understanding the functional contribution of O-GlcNAc elucidates the essential roles this modification plays in maintaining nutrient homeostasis and cellular signaling.

### SUMMARY OF INVENTION

To uncover the functions of the O-GlcNAc modification on a protein in cells, methods to either globally regulate O-GlcNAc or investigate specific glycosites have been developed (Worth, M., Li, H. & Jiang, J. Deciphering the Functions of Protein O-GlcNAcylation with Chemistry. ACS Chem Biol 2017, 12, 326-335). Over-expression, genetic knockdown/knockout, or application of chemical inhibitors for OGT or OGA are common mechanisms to globally elevate or reduce O-GlcNAc, respectively (Martin, S. E. S. et al. Structure-Based Evolution of Low Nanomolar O-GlcNAc Transferase Inhibitors. J. Am. Chem. Soc. 2018, 140, 13542-13545; Zhang, Z., Tan, E. P., VandenHull, N. J., Peterson, K. R. & Slawson, C. O-GlcNAcase Expression is Sensitive to Changes in O-GlcNAc Homeostasis. Front Endocrinol (Lausanne) 2014, 5, 206; Yuzwa, S. A. et al. A potent mechanism-inspired O-GlcNAcase inhibitor that blocks phosphorylation of tau in vivo. Nat. Chem. Biol. 2008, 4, 483-90). However, these approaches produce wide-spread changes to O-GlcNAc levels that require additional studies to characterize the function of O-GlcNAc on a target protein. Furthermore, inhibitors of OGT and OGA have recently been shown to rapidly induce abnormal expression of OGT (Martin, S. E. S. et al. Structure-Based Evolution of Low Nanomolar O-GlcNAc Transferase Inhibitors. J. Am. Chem. Soc. 2018, 140, 13542-13545) or OGA (Zhang, Z., Tan, E. P., VandenHull, N. J., Peterson, K. R. & Slawson, C. O-GlcNAcase Expression is Sensitive to Changes in O-GlcNAc Homeostasis. Front Endocrinol (Lausanne) 2014, 5, 206). With the advent of glycoproteomics methods (Alfaro, J. F. et al. Tandem mass spectrometry identifies many mouse brain O-GlcNAcylated proteins including EGF domain-specific O-GlcNAc transferase targets. Proc Natl Acad Sci USA 2012, 109, 7280-5; Vosseller, K. et al. O-linked N-acetylglucosamine proteomics of postsynaptic density preparations using lectin weak affinity chromatography and mass spectrometry. Mol. Cell. Proteomics 2006, 5, 923-34; Woo, C. M., Iavarone, A. T., Spiciarich, D. R., Palaniappan, K. K. & Bertozzi, C. R. Isotope-targeted glycoproteomics (IsoTaG): a mass-independent platform for intact N- and O-glycopeptide discovery and analysis. Nat. Methods 2015, 12, 561-7), specific glycosites are more readily targeted by site-directed mutagenesis approaches to permanently add O-GlcNAc (Gorelik, A. et al. Genetic recoding to dissect the roles of site-specific protein O-GlcNAcylation. Nat. Struct. Mol. Biol. 2019, 26, 1071-1077) or block its installation. However, using site-directed mutagenesis to study O-GlcNAcylated proteins with multiple or unmapped glycosites remains challenging. Additionally, O-GlcNAc has extensive cross-talk with other PTMs, including phosphorylation (Hart, G. W., Slawson, C., Ramirez-Correa, G. & Lagerlof, O. Cross talk between O-GlcNAcylation and phosphorylation: roles in signaling, transcription, and chronic disease. Annu. Rev. Biochem. 2011, 80, 825-58) and ubiquitylation (Ruan, H. B., Nie, Y. & Yang, X. Regulation of protein degradation by O-GlcNAcylation: crosstalk with ubiquitination. Mol. Cell. Proteomics 2013, 12, 3489-97), which may be disrupted by site-directed mutagenesis. An alternative method to selectively edit protein O-GlcNAcylation in cells facilitates dissection of O-GlcNAc functions on the target protein.

The selective installation of O-GlcNAc to a target protein in living cells using a nanobody fusion to OGT has been reported (Ramirez, D. H. et al. Engineering a Proximity-Directed O-GlcNAc Transferase for Selective Protein O-GlcNAcylation in Cells. ACS Chem Biol 2020, 15(4), 1059-1066). A nanobody is a small, single-domain antibody that is capable of binding to intracellular targets with high affinity and selectivity (Ingram, J. R., Schmidt, F. I. & Ploegh, H. L. Exploiting Nanobodies' Singular Traits. Annu. Rev. Immunol. 2018, 36, 695-715). In particular, nanobodies against green fluorescent protein (GFP) have been well-characterized and widely used for targeting GFP-tagged proteins and recruiting enzymes for protein-selective manipulation (Kirchhofer, A. et al. Modulation of protein properties in living cells using nanobodies. Nat. Struct. Mol. Biol. 2010, 17, 133-8). Building on the insights gained from engineering nanobody-OGT and the recent crystal structures of human OGA (Li, B., Li, H., Lu, L. & Jiang, J. Structures of human O-GlcNAcase and its complexes reveal a new substrate recognition mode. Nat. Struct. Mol. Biol. 2017, 24, 362-369; Roth, C. et al. Structural and functional insight into human O-GlcNAcase. Nat. Chem. Biol. 2017, 13, 610-612; Elsen, N. L. et al. Insights into activity and inhibition from the crystal structure of human O-GlcNAcase. Nat. Chem. Biol. 2017, 13, 613-615), the present disclosure describes the development of an “eraser” to remove O-GlcNAc from a desired target protein in cells by leveraging the protein selectivity of the nanobody and the robust enzymatic activity of OGA after rational engineering (FIG. 1A). The functional domains for OGA were identified and the feasibility of fusing a nanobody for targeted deglycosylation on nucleoporin 62 (Nup62) in cells was verified. A split OGA with minimal size and limited inherent activity on protein substrates was developed, and the ability of a nanobody fused to split OGA to restore deglycosidase activity selectively to the target protein Nup62 was demonstrated (FIG. 1B). A nanobody against GFP (nGFP) fused to split OGA was utilized to remove O-GlcNAc from a series of GFP-tagged proteins in living cells. The selectivity of the targeted O-GlcNAc eraser was validated by a quantitative proteomic analysis of the O-GlcNAc proteome. The present disclosure demonstrates a general and facile mechanism to “erase” (remove)O-GlcNAc from a desired target protein, which can be used to engineer and decipher the specific functions of O-GlcNAc within cells.

The present disclosure provides methods to selectively remove O-GlcNAc and measure the functional contribution on a desired target protein using a nanobody-splitOGA. The targeted O-GlcNAc “erasers” enable manipulation of O-GlcNAc levels on a protein in cells, and complements chemical and genetic methods to globally perturb O-GlcNAc levels or target specific glycosites, if known. To develop the targeted O-GlcNAc erasers, activities of several truncated OGA constructs on GFP-Nup62 were examined in vivo. Both the catalytic domain and parts of the stalk domain are essential for deglycosidase activity within cells, presumably because of the formation of the unusual homodimer revealed by the OGA structure (Li, B., Li, H., Lu, L. & Jiang, J. Structures of human O-GlcNAcase and its complexes reveal a new substrate recognition mode. Nat. Struct. Mol. Biol. 2017, 24, 362-369; Roth, C. et al. Structural and functional insight into human O-GlcNAcase. Nat. Chem. Biol. 2017, 13, 610-612; Elsen, N. L. et al. Insights into activity and inhibition from the crystal structure of human O-GlcNAcase. Nat. Chem. Biol. 2017, 13, 613-615). However, truncated OGA maintains high deglycosidase activity and therefore yields certain off-target effects on non-targeted proteins. To improve selectivity, the naturally existing caspase-3 cleavage site on OGA can be utilized to generate a split OGA with reduced substrate activity. For example, by optimization of a series of N and C fragments, minimal split OGA fragments N2 and C3 were found to possess limited deglycosidase activity unless tethered to a nanobody (e.g., nGFP), which restored deglycosidase activity selectively to the target protein. The protein-selective nature of O-GlcNAc erasers is demonstrated by the ability to selectively deglycosylate several GFP-tagged glycoproteins when evaluated in HEK 293T cells, including Nup62, Sp1, JunB and c-Jun, proteins representative of the range of O-GlcNAc levels and substrates. Nanobody-split OGAs have successfully achieved O-GlcNAc removal on target proteins with little global perturbation, as confirmed by examining endogenous glycoprotein CREB and the global glycoproteome.

The protein-selective O-GlcNAc erasers disclosed herein can selectively remove O-GlcNAc from proteins and can complement existing methods to reduce O-GlcNAc globally or via site-directed mutagenesis in cells. The protein-selective O-GlcNAc erasers disclosed herein can edit O-GlcNAc on desired proteins in situ within cells. The nanobody-splitOGA system is a generalizable and flexible strategy for targeting protein deglycosylation and is composed of three (or more) modules, portions, or pieces, including but not necessarily limited to: nanobody, N-terminal fragments, and C-terminal fragments. Recruitment through a variety of alternative nanobodies can also be used, for example nanobodies that target various tags, such as EPEA-tag31, SPOT-TAG® (Traenkle, B. et al. Monitoring interactions and dynamics of endogenous beta-catenin with intracellular nanobodies in living cells. Mol. Cell. Proteomics 2015, 14, 707-23). Optimal nanobodies can be generated using robust platforms (English, J. G. et al. VEGAS as a Platform for Facile Directed Evolution in Mammalian Cells. Cell 2019, 178, 748-761 e17), and can be used to target endogenous protein substrates. Nanobodies with a wide range of affinities can be used to tune the catalytic turnover of nanobody-splitOGA, (Fridy, P. C. et al. A robust pipeline for rapid production of versatile nanobody repertoires. Nat. Methods 2014, 11, 1253-60), or chemo/optogenetically controlled nanobodies can be utilized (Gil, A. A. et al. Optogenetic control of protein binding using light-switchable nanobodies. bioRxiv (2019), https://doi.org/10.1101/739201). These other nanobodies are understood to be useful in the nanobody-splitOGAs of the disclosure in order to generate additional versions of an O-GlcNAc eraser with greater spatiotemporal control.

The targeted O-GlcNAc erasers of the present disclosure can offer a lower-barrier approach to evaluating the function of O-GlcNAc on a target protein in the absence of access to glycosite maps for mutagenesis. For example, hyper-O-GlcNAcylation is a general feature of cancer linked to various phenotypes (Ma, Z. & Vosseller, K. Cancer metabolism and elevated O-GlcNAc in oncogenic signaling. J. Biol. Chem. 2014, 289, 34457-65), and changes in nutrient condition and cellular stress drive the fluctuation of O-GlcNAcylation levels on multiple protein substrates along these pathways more broadly. The contribution of individual O-GlcNAcylated proteins can be individually targeted using a nanobody-splitOGA. Because the nanobody-splitOGA system relies on the same hydrolase activity as fl-OGA, the effect of O-GlcNAc and potential cross-talk with other tailoring post-translational modifications is possible. The nanobody-splitOGA together with the nanobody-OGT17, can selectively modulate O-GlcNAcylation status and facilitate the attribution of unique functions to specific proteins, delineating the cause and effect in a loss-of-function or gain-of-function manner, respectively. O-GlcNAc erasers can also provide a potential avenue for engineering intracellular behaviors through O-GlcNAc and regulation of signaling transduction for therapeutic intervention in the long-term (Ong, Q., Han, W. & Yang, X. O-GlcNAc as an Integrator of Signaling Pathways. Front Endocrinol (Lausanne) 2018, 9, 599).

The present disclosure provides for targeted O-GlcNAc erasers utilizing a nanobody fused to a split OGA to execute selective O-GlcNAc hydrolysis on target protein in living cells. This strategy can result in the successful removal of O-GlcNAc from specific glycoproteins while leaving the broader O-GlcNAc proteome largely intact. For example, the targeted O-GlcNAc erasers enabled the observation of a direct link between O-GlcNAcylation on c-Jun. Protein selective O-GlcNAc erasers can be extended to measure the many functions of O-GlcNAc on other proteins of interest, and will allow the observation and rewiring of the signaling network in living cells in the future.

In one aspect, the present disclosure provides fusion proteins comprising a nanobody, or fragment thereof, and a split glycosyl hydrolase comprising more than one piece, where the nanobody is fused to at least one piece of the split glycosyl hydrolase. In some aspects, the split glycosyl hydrolase is a split O-GlcNAcase (OGA). In some aspects, the split glycosyl hydrolase comprises (i) a first piece comprising a catalytic domain, and (ii) a second piece comprising a stalk domain. In other aspects, either the catalytic domain or the stalk domain, or both, is truncated. In other aspects, the catalytic domain comprises amino acid residues 1-400 of SEQ ID NO: 1, or the stalk domain comprises amino acid residues 544-706 of SEQ ID NO: 1, or both. In another aspect, the present disclosure provides that the nanobody is fused to the N terminus of a piece that comprises a stalk domain. The nanobody can bind to a cell surface protein. The nanobody can bind to GFP, EPEA, or UBC6e. In other embodiments, the nanobody and at least one piece of the split glycosyl hydrolase are fused via a peptide linker.

In one aspect, the present disclosure provides a split OGA enzyme comprising (i) a first piece comprising a truncated catalytic domain, and (ii) a second piece comprising a truncated stalk domain.

In one aspect, the present disclosure provides a pharmaceutical composition comprising the fusion protein. In another aspect, the present disclosure provides a polynucleotide encoding the fusion protein. In one aspect, the present disclosure provides a vector comprising a polynucleotide encoding a fusion protein. In another aspect, the present disclosure provides a cell comprising a fusion protein. In one aspect, the present disclosure provides a cell comprising the nucleic acid molecule encoding a fusion protein.

Also provided in the present disclosure are methods of use, which involve a fusion protein disclosed herein. In one aspect, the present disclosure provides a method of removing a sugar from a target protein, the method comprising contacting a protein with a sugar moiety with a fusion protein, thereby removing the sugar moiety from the protein. In another aspect, the present disclosure provides a method of studying the effect of glycosylation on protein function in a cell using a fusion protein disclosed herein. In other aspect, the present disclosure provides the removed sugar moiety is an O-linked N-acetyl glucosamine. In other aspects, the target protein is a transcription factor or a nucleoporin, including but not limited to transcription factors is selected from the group consisting of Sp1, JunB, and c-Jun or wherein the nucleoporin is Nup62.

In another aspect, the present disclosure provides a kit comprising any of the fusion proteins, pharmaceutical compositions, polynucleotides, vectors, or cells disclosed herein.

The present disclosure also provides methods of treating a subject. In one aspect, the present disclosure provides a method of treating a disease or disorder (e.g., neurodegenerative diseases (Parkinson's disease, Huntington's disease, Alzheimer's disease, dementia, multiple system atrophy), cancer, and diabetes), the method comprising administering a fusion protein to a subject in need thereof. In one aspect, the present disclosure provides a method of treating a subject suffering from or susceptible to a neurodegenerative disease, the method comprising administering an effective amount of a fusion protein to the subject. In another aspect, the present disclosure provides a method of treating a subject suffering from or susceptible to cancer, the method comprising administering an effective amount of a fusion protein to the subject. In one aspect, the present disclosure provides a method of treating a subject suffering from or susceptible to diabetes, the method comprising administering an effective amount of a fusion protein to the subject.

The details of certain embodiments of the invention are set forth in the Detailed Description of Certain Embodiments, as described below. Other features, objects, and advantages of the invention will be apparent from the Definitions, Figures, Examples, and Claims.

### BRIEF DESCRIPTION OF THE DRAWINGS

FIGS. 1A-1B demonstrate the design and development of a nanobody-directed split OGA for O-GlcNAc removal in a protein-selective manner. FIG. 1A shows a schematic of an approach for target protein deglycosylation using a nanobody-directed O-GlcNAc eraser. The nanobody, like nGFP, is able to recognize a certain target, like GFP, and redirect the enzyme to erase the O-GlcNAc modification on the target protein. FIG. 1B shows a schematic of the engineered OGA to achieve protein selectivity. OGA was engineered into a split and truncated form with limited inherent substrate activity. The fusion of nanobody to the engineered OGA promoted localization to and deglycosylation of the desired target protein. The N and C fragments shown here are termed N2 and C3, respectively.

FIGS. 2A-2D demonstrate the design and optimization of nanobody-fused split OGA for protein-selective deglycosylation on GFP-Nup62. FIG. 2A shows a schematic of the split site, N fragments, and C fragments tested in this study. Amino acid numbers appear on top. The optimal combination N2 with nGFP-fused C3 (shown in dotted rectangle) is termed nGFP-splitOGA. FIG. 2B shows an immunoblot representing the optimization of the N-fragment with C1 on GFP-Nup62 to obtain a minimal N-fragment. GFP-Nup62 was co-expressed with the indicated constructs, enriched by anti-EPEA beads, and analyzed by immunoblotting to reveal the protein level and O-GlcNAc modification level respectively. Expression of the indicated proteins and O-GlcNAc modification were analyzed by immunoblotting. FIG. 2C shows an immunoblot representing the optimization of the C-fragment with N2 on GFP-Nup62 to obtain a minimal C-fragment with limited inherent substrate activity. GFP-Nup62 was co-expressed with the indicated constructs, enriched by anti-EPEA beads, and analyzed by immunoblotting to reveal the protein level and O-GlcNAc modification level respectively. Expression of the indicated proteins and O-GlcNAc modification were analyzed by immunoblotting. FIG. 2D shows an immunoblot representing that the nGFP fusion to split OGA regains activity on GFP-Nup62. The optimized split OGA (N2+C3) shows limited substrate activity that is reinstated after fusion with nGFP on either fragment. GFP-Nup62 was co-expressed with the indicated constructs, enriched by anti-EPEA beads, and analyzed by immunoblotting to reveal the protein level and O-GlcNAc modification level respectively. Expression of the indicated proteins and O-GlcNAc modification were analyzed by immunoblotting. fl-OGA, full-length OGA. WCL, whole cell lysate.

FIGS. 3A-3G demonstrate that the nanobody-fused split OGA is general for protein-selective deglycosylation on various O-GlcNAcylated proteins. Deglycosidase activity of fl-OGA and the split OGA (N2+C3) with or without fusion of nGFP on GFP-tagged Nup62 (FIG. 3A), Sp1 (FIG. 3B), and JunB (FIG. 3C). In FIGS. 3A-3B, GFP-tagged proteins were co-expressed with the indicated protein then enriched by anti-EPEA beads and analyzed by immunoblotting. Ratios are quantified from the protein level and O-GlcNAc modification level. In FIG. 3C, cell lysates under different conditions were subjected to chemoenzymatic labeling followed by a mass shift assay with 5 kDa DBCO-PEG, detected using antibodies against Flag and endogenous CREB. O-GlcNAcylation levels were measured by the ratio between the intensity of mass-shifted bands and unmodified bands. Representative immunoblots are shown. The graph is shown as mean±s.d. of n=3 independent experiments. Unpaired two-tailed Student's t tests were used for statistical analysis in FIGS. 3A-3C. P values were (FIG. 3A) 6.70×10⁻⁵ (****), 3.45×10⁻⁴ (***), (FIG. 3B) 1.00×10⁻⁶ (****) for treatments versus blank, (FIG. 3C) 7.36×10⁻³ (**) for JunB versus CREB, n.s., not significant. FIG. 3D shows log 2 ratios of changes of enriched O-GlcNAcylated protein abundance of fl-OGA and nGFP-splitOGA versus nGFP-splitOGA(D174N)-treated cells respectively. The first and second groups show identified proteins without GFP-Nup62. The solid lines refer to the median of each group. FIG. 3E shows log 2 ratios of changes of enriched O-GlcNAcylated protein abundance of nGFP-splitOGA versus fl-OGA-treated cells. Each dot represents the ratio of an individual protein from the average of two biological independent replicates. GFP-Nup62 was indicated by the labeled dot. Symbols represent the corresponding OGA constructs as indicated. fl-OGA, full-length OGA. FIGS. 3F-3G shows volcano plots illustrating the comparison of enriched O-GlcNAcylated proteins of nGFP-splitOGA (FIG. 3F) or of inactive (FIG. 3G) versus split OGA-treated cells with co-expression of GFP-Sp1. P=0.05 and grey-dashed lines denote ±1.5-fold change as the significance threshold. Each point represents an individual identified protein of two (FIGS. 3D-3E) or four (FIGS. 3F-3G) independent biological replicates. GFP-Nup62 or GFP-Sp1 are indicated by the labeled dots. Symbols represent the corresponding OGA constructs as indicated. A two-tailed, unpaired Student's t-test was used for statistical analysis in FIGS. 3C, 3F, and 3G. n.s., not significant.

FIGS. 4A-4C demonstrate that a variety of alternative nanobodies can be used in the split OGA system for targeted protein deglycosylation. FIG. 4A provides a schematic of nanobodies against the EPEA and 14-residue Ubc tag adapted to split OGA. SEQ ID NO: 11 (QADQEAKELARQIA) is shown. FIG. 4B shows a schematic and immunoblot of selective removal of O-GlcNAc from Nup62 tagged with Ubc and EPEA using nEPEA-splitOGA or nUbc-splitOGA. Anti-myc and anti-HA blots detect expression of N2 and nanobody-C3, respectively. Results are representative of two biological replicates. FIG. 4C shows a schematic of an immunoblot with an anti-O-GlcNAc antibody to evaluate the O-GlcNAc modification level. Treatment with the nUbc-containing fusion protein significantly lowered the level of O-GlcNAc modification. c-Fos with Ubc tag can be selectively deglycosylated by nUbc-splitOGA. Representative immunoblots are shown. Quantitative results are the mean±s.d. of n=3 independent experiments, using unpaired two-tailed Student's t tests for statistical analysis. n.s., not significant. WCL, whole cell lysate.

FIGS. 5A-5E demonstrate that the nanobody-fused split OGA reveals the direct correlation between O-GlcNAc on c-Jun and protein stability and heterodimerization with c-Fos. FIG. 5A shows that nGFP-splitOGA can remove O-GlcNAc from GFP-c-Jun selectively. A mass shift assay was conducted to evaluate the O-GlcNAc level on both GFP-c-Jun and endogenous CREB. The O-GlcNAcylation levels were measured by the ratio between the intensity of mass-shifted bands and unmodified bands. Representative immunoblots are shown. FIG. 5B shows that the stability of GFP-c-Jun is directly related to the extent of O-GlcNAc modification. GFP-c-Jun and nGFP-splitOGA or [N2(D174N)+nGFP-C3] were expressed in HEK 293T cells simultaneously. Cells were incubated with 50 μM CHX for up to 12 h and monitored for GFP-c-Jun at different time points. The graph is shown as mean±s.d. of n=3 independent experiments. Unpaired two-tailed Student's t tests were used for statistical analysis in FIGS. 5A-5B. P values were (FIG. 5A) 2.12×10⁻² (*) for c-Jun versus CREB, (FIG. 5B) 2.25×10⁻² (*) between two groups at 12 h, n.s., not significant. FIG. 5C shows a schematic and immunoblot demonstrating that c-Fos-Ubc-Flag-EPEA was deglycosylated by both nUbc-split OGA and OGT inhibition. nUbc-split OGA displayed good selectivity and showed a negligible effect on other O-GlcNAcylated proteins globally, in contrast to OGT inhibition, which displayed poor selectivity. FIGS. 5D-5E show an AP-1 luciferase assay showing transcription activity upon OGT inhibition by OSMI-4b (FIG. 5D), or upon the expression of nUbc-splitOGA (FIG. 5E) in c-Fos-Ubc co-transfected HEK 293T cells.

FIGS. 6A-6D provide a schematic representation of exemplary constructs for OGA the target proteins-of-interest. FIG. 6A provides a schematic of the structures of human OGA and other truncations. The catalytic domain, stalk domain, HAT domain, and intrinsic disordered regions are shown. The GS linker represents a 15-residue glycine and serine linker. SEQ ID NO: 12 (GSGSGSGSGSGSGSG) is shown. FIG. 6B provides a depiction of the strategy used to fuse the nanobody on OGAs to achieve protein specificity. nGFP, nanobody against GFP. FIG. 6C shows the design of GFP-fused, Ubc tag-fused, and BC2 tag-fused proteins of interest used in this study. For GFP-fused proteins, GFP and a Flag tag are placed on the N-terminus, and the EPEA tag is on the C-terminus unless otherwise noted. For Ubc tag-fused proteins, the 14-residue peptide tag, Flag tag, and EPEA tag are sequentially placed in the C-terminus unless otherwise noted. For BC2 tag-fused proteins, the 12-residue peptide tag is placed on the N-terminus, and the Flag and EPEA tags are on the C-terminus. Peptide sequences of Ubc and BC2 are provided. SEQ ID NOs: 11 (QADQEAKELARQIA) and 13 (PDRKAAVSHWQQ) are shown. FIG. 6D shows the symbols used herein to represent the indicated split OGA constructs.

FIGS. 7A-7B show the identification of the minimal OGA for nanobody-directed deglycosylation on a target protein. FIG. 7A shows the evaluation of the enzymatic activities of OGA and its truncations on GFP-Nup62. GFP-Nup62 was co-expressed with the indicated constructs, enriched by anti-EPEA beads, and analyzed by immunoblotting to visualize the protein level and O-GlcNAc modification level, respectively. FIG. 7B shows the evaluation of the enzymatic activities of nGFP-OGA fusion proteins on GFP-Nup62. Expression levels of the indicated proteins and degree of O-GlcNAc modification were quantified by immunoblotting. The ratio is equal to the intensity of anti-O-GlcNAc immunoblot normalized by the intensity of anti-Flag immunoblot. WCL, whole cell lysate. The data are representative of two biological replicates.

FIGS. 8A-8C show that nGFP-OGA(GS-AHAT) has limited target protein selectivity and can alter subcellular localization of the target protein. FIG. 8A shows that nGFP-OGA(GS-AHAT) removes O-GlcNAc from Nup62 without a GFP tag similar to the full length OGA (fl-OGA). HEK 293T whole cell lysates (WCL) and immunoprecipitation samples were analyzed by immunoblotting assays using the indicated antibodies. Results are representative of two biological replicates. FIG. 8B shows that nGFP colocalizes with nuclear transcription factor Sp1 fusion with GFP and does not change the subcellular localization of GFP-Sp1 by immunofluorescence imaging. FIG. 8C shows that nGFP-OGA(GS-AHAT) alters the subcellular localization of GFP-Sp1, but co-expression with fl-OGA does not change nuclear localization of GFP-Sp1 by immunofluorescence imaging. Channels are annotated on the top. Scale bar: 10 μm. Right: merged channel. Proteins co-expressed in each sample are labeled on the left side. Images are representative of at least three randomly selected frames.

FIGS. 9A-9C show that optimization of nGFP-fused split OGA constructs in living cells. FIG. 9A shows that co-expression of N- and C-fragments of OGA reconstitutes deglycosidase activity in HEK 293T cells. FIG. 9B shows that split OGA fragments, N2 and C3, instead of C4, associate with each other when co-expressed in HEK 293T cells. The asterisk indicates IgG heavy chain from anti-c-Myc magnetic beads. FIG. 9C provides a comparison of nGFP-fused N- and C-terminal OGA fragments on GFP-Nup62 in HEK 293T cells. The pair of N2 and nGFP-fused C3 (N2+nGFP-C3) shows the best deglycosylation performance. In FIG. 9A and FIG. 9C, activities of fragments alone or pairs with/without nGFP were evaluated on GFP-Nup62, which was enriched by beads against EPEA tag and blotted with RL2 antibody to reveal O-GlcNAc modification level. D174N is a catalytically impaired mutation on OGA. Anti-myc and anti-HA blots detect expression of full-length (fl-OGA) or N-terminal fragment, and C-terminal fragment, respectively. WCL, whole cell lysates. The data in FIGS. 9A-9C are representative of at least two biological replicates.

FIGS. 10A-10D show that nGFP-splitOGA selectively deglycosylates the target protein without affecting the global O-GlcNAc proteome. FIG. 10A shows that nGFP-splitOGA has little effect on endogenous glycoprotein CREB. HEK 293T cells co-expressing OGA constructs with GFP-Nup62 were subjected to mass shift assay. The intensities of O-GlcNAcylated and unmodified CREB were quantified. The ratios are shown below the anti-CREB blot. WCL, whole cell lysates. FIG. 10B shows that overexpression of selected split OGA constructs with target protein has little effect on global O-GlcNAcylation levels. For OGT inhibition, cells were treated with 25 μM OSMI-4b for 30 h. Global O-GlcNAcylation level was evaluated by anti-O-GlcNAc (RL2) antibody. FIG. 10C shows that nGFP-splitOGA has minimal effect on protein levels of endogenous OGT, OGA, and glycoprotein CREB. Anti-myc and anti-HA blots detect expression of N-terminal fragment, and C-terminal fragment, respectively. FIG. 10D provides a comparison of overexpressed proteins with the corresponding endogenous proteins. The antibody against OGA recognizes both endogenous OGA and the overexpressed N-terminal fragment of split OGA. Endogenous Nup62(*) and OGA (**) are indicated. The data in FIGS. 10A-10D are representative of at least two biological replicates.

FIGS. 11A-11C show confocal imaging of intracellular distributions of GFP-Sp1 and the split OGAs in HEK 293T cells. FIG. 11A shows GFP-Sp1 localizes in the nucleus. FIG. 11B shows intracellular distributions of N2 and nGFP-C3 fragments when co-expressed in HEK 293T cells. The two fragments of nGFP-splitOGA were distributed in both the cytoplasm and the nucleus. FIG. 11C shows subcellular localizations of the GFP-Sp1 N fragment and C fragment when expressed simultaneously in HEK 293T cells. Two fragments of split OGA without nGFP (FIG. 11C, upper row) were distributed in both the cytoplasm and nucleus. The C-terminal fragment of nGFP-splitOGA (FIG. 11C, bottom row) reveals better colocalization with nuclear protein GFP-Sp1, showing the binding between nGFP and GFP. Split OGAs do not change the subcellular localization of GFP-Sp1. Channels are annotated on the top. Scale bar: 10 μm. Right: merged channel. Proteins co-expressed in each sample are labeled on the left side. Images are representative of at least three randomly selected frames.

FIGS. 12A-12D show mass spectrometry analysis on the activity and selectivity of nGFP-splitOGA on GFP-Nup62. FIG. 12A provides a schematic representation of the workflow of O-GlcNAcylated protein enrichment and mass spectrometry-based identification. Proteins with O-GlcNAc modification were labeled with GalNAz by GalT(Y289L)-mediated chemoenzymatic labeling, followed by a click reaction with an alkyne-biotin probe. Biotin-labeled proteome was enriched by streptavidin beads and digested by trypsin. Released peptides were labeled by TMT reagents and compiled into a single pool. Proteins were identified and quantified by LC-MS. FIGS. 12B-12D demonstrate the reproducibility of the TMT experiments of O-GlcNAcylated proteome shown in FIGS. 3D-3E. The signal abundances of the corresponding TMT channels for each protein were extracted and were log 10 transformed for full-length OGA treatment (FIG. 12B, fl-OGA), nGFP-splitOGA treatment (FIG. 12C) and its inactive form [N2(D174N)+nGFP-C3] treatment (FIG. 12D) groups (n=2 independent biological replicates).

FIGS. 13A-13B show that peptide tag BC2 and its nanobody can be adapted to split OGA to achieve protein-selective deglycosylation. FIG. 13A provides a schematic of nanobodies against BC2 and EPEA tag adapted to split OGA. BC2 tag refers to a 12-residue peptide epitope, which is functional irrespective of its position on the target protein. SEQ ID NO: 13 (PDRKAAVSHWQQ) is shown. FIG. 13B shows that nBC2-splitOGA is able to remove O-GlcNAc from Nup62 tagged with BC2 and EPEA in a similar manner to nEPEA-splitOGA. Symbols represent the corresponding OGA constructs as indicated in FIG. 6. Anti-myc and anti-HA blots detect expression of full-length (fl-OGA) or N-terminal and C-terminal fragment, respectively. WCL, whole cell lysate. The data are representative of two biological replicates.

FIGS. 14A-14C show modulation of O-GlcNAc modification level and validation of its functional contributions on the stability of GFP-c-Jun. FIG. 14A shows evaluation of O-GlcNAc level on GFP-c-Jun and endogenous CREB by the mass-shift assay. GFP-c-Jun was co-expressed with the indicated OGA constructs. The intensities of O-GlcNAcylated and unmodified c-Jun and CREB were quantified. Quantification is shown as mean±s.d. of n=3 independent biological experiments. All ratios were normalized by the blank samples. Unpaired two-tailed Student's t tests were used for statistical analysis. n.s., not significant. FIG. 14B shows analysis of whole cell lysates (WCL) by immunoblotting assays using the indicated antibodies. Anti-myc and anti-HA blots detect expression of full-length (fl-OGA) or N-terminal fragment, and C-terminal fragment, respectively. The data in FIGS. 14A and 14B are representative of at least three biological replicates. FIG. 14C shows that the stability of GFP-c-Jun was enhanced by OGA inhibition (Thiamet-G treatment) and impeded by OGT inhibition (Ac45SGlcNAc treatment). HEK 293T cells expressing GFP-c-Jun pre-treated with DMSO or Ac45SGlcNAc or Thiamet-G were incubated with 50 μM CHX for up to 12 h, during which the protein level of GFP-c-Jun and global O-GlcNAcylation level were monitored. Results in FIG. 14C are representative of two biological replicates.

FIGS. 15A-15B show modulation of O-GlcNAc modification level with nUbc-splitOGA on c-Fos-Ubc in comparison to OGT inhibition. Immunoblotting analysis of protein expression and O-GlcNAcylation status of c-Fos-Ubc and endogenous c-Jun under the indicated treatments corresponding to FIGS. 5D-5E are shown by either enrichment against EPEA-tag (FIG. 15A) or chemoenzymatic labeling followed with Biotin-IP (FIG. 15B). Endogenous c-Jun shows negligible changes on O-GlcNAcylation status with the co-expression of nUbc-splitOGA but shows reduced O-GlcNAc modification upon OGT inhibition with OSMI-4b. No detectable endogenous c-Fos was observed in HEK 293T cells. The data in FIGS. 15A and 15B are representative of two biological replicates.

FIGS. 16A-16C show immunoblotting analysis results of the whole cell lysates from the experiments shown in FIGS. 2B-2D. Immunoblotting analysis of global O-GlcNAc level on the whole cell lysates from FIG. 2B (FIG. 16A), FIG. 2C (FIG. 16B) and FIG. 2D (FIG. 16C), respectively, using the indicated antibodies is shown. The data in FIGS. 16A-16C are representative of two biological replicates.

FIG. 17 shows screening of various C-fragments paired with the shortest N-fragment, N3, with or without nGFP. N3 contains only the catalytic domain (residues 1-366, OGA(cat), shown in FIG. 2A). GFP-Nup62 was enriched by beads against EPEA tag and blotted with O-GlcNAc antibody to reveal O-GlcNAc modification level. N3 is unable to reconstitute OGA's activity with any C-fragments shown in this study even in the presence of nGFP. In these experiments, whole cell lysates (WCL) were analyzed by immunoblotting assays using the indicated antibodies. Anti-myc and anti-HA blots detect expression of full-length (fl-OGA) or N-terminal fragment and C-terminal fragment, respectively. The data are representative of two biological replicates.

FIG. 18 shows that Nup62 with a C-terminal GFP can be targeted and deglycosylated by nGFP-splitOGA in HEK 293T cells. Nup62 with GFP and a Flag-tag at the C-terminus was co-expressed with myc-OGA or nGFP-splitOGA, which was immunoprecipitated using ANTI-FLAG® M2 Magnetic Beads and blotted with anti-O-GlcNAc antibody. In these experiments, whole cell lysates (WCL) were analyzed by immunoblotting assays using the indicated antibodies. Anti-myc and anti-HA blots detect expression of the full-length (fl-OGA) or N-terminal fragment, and C-terminal fragment, respectively. The data are representative of two biological replicates.

FIGS. 19A-19C show quantitative results and immunoblotting analysis of the whole cell lysates from the experiments shown in FIGS. 3A-3C. Quantitative results of immunoblots shown in FIG. 3A (FIG. 19A), FIG. 3B (FIG. 19B) are the mean±s.d. of n=3 independent experiments, using unpaired two-tailed Student's t tests for statistical analysis. Whole cell lysates were analyzed by immunoblotting assays using the indicated antibodies. Anti-myc and anti-HA blots detect expression of the full-length (fl-OGA) or N-terminal fragment and C-terminal fragment, respectively. Endogenous CREB was monitored under the indicated conditions in FIG. 3C. Results in FIGS. 19A-19C are representative of three biologically independent experiments.

FIGS. 20A-20C show confocal imaging of subcellular localization of target proteins with Ubc tag and nUbc-splitOGA used in this study in HEK 293T cells. FIG. 20A shows that two fragments of nUbc-splitOGA were distributed on both cytoplasm and nucleus when co-expressed in cells. FIGS. 20B-20C show subcellular localizations of Nup62-Ubc (FIG. 20B), c-Fos-Ubc (FIG. 20C), N fragment and C fragment when expressed simultaneously in HEK 293T cells. nUbc-C3 reveals better colocalization with Nup62-Ubc (FIG. 20B) and c-Fos-Ubc (FIG. 20C). In FIGS. 20B and 20C, Ubc tag-fused proteins were labeled by DYKDDDDK (SEQ ID NO: 14) Tag (D6W5B) rabbit primary antibody and Alexa Fluor™ 488 anti-rabbit secondary antibody. C-terminal fragment with a HA tag was labeled by anti-HA-Tag (Alexa Fluor® 647 Conjugate). N-terminal fragment with a myc tag was labeled by Myc-Tag (9B11) mouse primary antibody and Alexa Fluor™ 568. The nucleus was stained with NucBlue™ Fixed Cell Stain ReadyProbes™ reagent. Channels are annotated on the top. Scale bar: 10 μm. Right: merged channel. Proteins expressed in each sample were annotated on the left side. Images are representative of at least three randomly selected frames.

FIG. 21 shows immunoblotting analysis results of the whole cell lysates from the experiments shown in FIG. 4C. Whole cell lysates (WCL) were analyzed by immunoblotting assays using the indicated antibodies. Anti-myc and anti-HA blots detect expression of the full-length (fl-OGA), N-terminal fragment, and C-terminal fragment, respectively. The results are representative of three biologically independent experiments.

FIGS. 22A-22B show additional immunoblotting analysis results of the experiment shown in FIG. 5A. In FIG. 22A, whole cell lysates (WCL) from the representative result in FIG. 5A were analyzed by immunoblotting assays using the indicated antibodies. FIG. 22B shows another representative biological replicate of the experiment shown on FIG. 5A. Anti-myc and anti-HA blots detect expression of the full-length (fl-OGA) or N-terminal fragment, and C-terminal fragment, respectively. Results in FIG. 22A and FIG. 22B are representative of at least three biologically independent experiments.

FIG. 23 shows that overexpression of different forms of split OGA constructs has little effect on global O-GlcNAcylation level. Split OGA and nanobody-fused split OGA constructs were expressed in HEK 293T cells as indicated. For OGT inhibition, cells were treated with 25 μM OSMI-4b for 30 h. Global O-GlcNAcylation level was evaluated by anti-O-GlcNAc (RL2) antibody. Anti-myc and anti-HA blots detect expression of the full-length (fl-OGA), N-terminal fragment, and C-terminal fragment, respectively. The data are representative of two biological replicates.

FIGS. 24A-24B show a comparison of deglycosylation by nanobody-splitOGA and by OGT inhibition with OSMI-4b on target proteins. HEK 293T cells were simultaneously transfected with (1) nanobody-splitOGA or its inactive mutant, and (2) target protein GFP-Nup62 (FIG. 24A) or GFP-Sp1 (FIG. 24B). For OGT inhibition, cells expressing target protein were treated with DMSO as the negative control or 25 μM OSMI-4b for 30 h. Target proteins were enriched by beads against EPEA tag and blotted with O-GlcNAc (RL2) antibody to reveal O-GlcNAc modification level. Whole cell lysates (WCL) were analyzed by immunoblotting assays using the indicated antibodies. Anti-myc and anti-HA blots detect expression of N-terminal fragment and C-terminal fragment, respectively. Major changes on global O-GlcNAcylation blots in the first and second lanes derive from deglycosylated GFP-Nup62 (FIG. 24A), GFP-Sp1 (FIG. 24B) and their truncated fragments. The data in FIGS. 24A and 24B are representative of two biological replicates.

FIGS. 25A-25B show confocal imaging of subcellular localization of target proteins with GFP or Ubc tag in HEK 293T cells. GFP-fused proteins were imaged under 488 nm excitement wavelength. Ubc tag-fused proteins were labeled by DYKDDDDK (SEQ ID NO: 14) Tag (D6W5B) rabbit primary antibody and Alexa Fluor™ 488 anti-rabbit secondary antibodyGFP. Nucleus was stained with NucBlue™ Fixed Cell Stain ReadyProbes™ reagentDAPI. Channels are annotated on the top. Scale bar: 10 μm. Right: merged channel. Proteins expressed in each sample were annotated on the left side. Images are representative of at least three randomly selected frames.

FIG. 26 shows that split OGA is unable to interact with OGT as the full-length OGA. OGT with His tag was co-transfected with different OGA constructs in HEK 293T cells as indicated. Overexpressed OGT and its interacting proteins were co-immunoprecipitated by beads against His tag. Whole cell lysates and co-immunoprecipitated proteins were analyzed by immunoblotting assays using the indicated antibodies. Anti-myc and anti-HA blots detect expression of full-length OGA (fl-OGA), N-terminal fragment, and C-terminal fragment, respectively. The data are representative of two biological replicates.

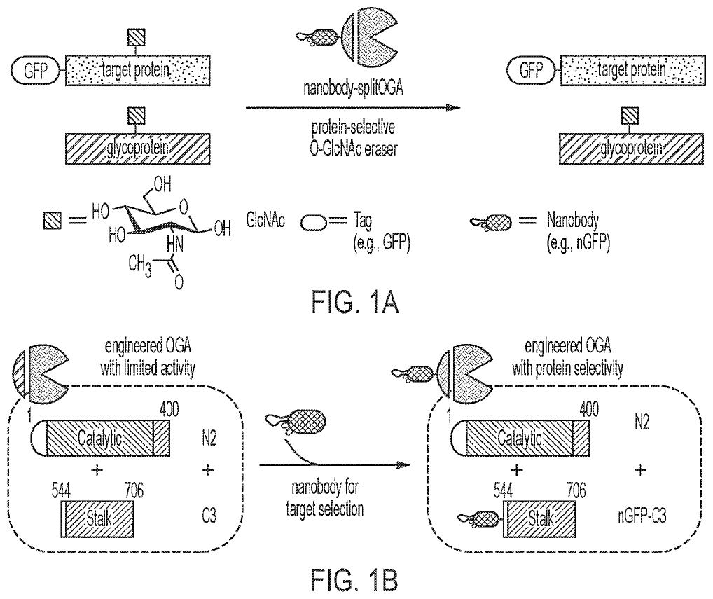

### DEFINITIONS

Descriptions and certain information relating to various terms used in the present disclosure are collected herein for convenience.

As used herein and in the claims, the singular forms “a,” “an,” and “the” include the singular and the plural reference unless the context clearly indicates otherwise. Thus, for example, a reference to “an agent” includes a single agent and a plurality of such agents.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may also be a “split protein.” A split protein, as used herein, refers to a protein that has been engineered to be expressed as two separate pieces. Together, the separate pieces may comprise the full-length protein, or they may comprise only a portion of the full-length protein.

A “nanobody,” as used herein, refers to a small protein recognition domain. Further, a nanobody is the smallest antigen binding fragment or single variable domain derived from naturally occurring heavy chain antibody and is known to the person skilled in the art. They are derived from heavy chain only antibodies, seen in camelids (Hamers-Casterman et al. 1993; Desmyter et al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). The single variable domain heavy chain antibody is herein designated as a nanobody or a VHH antibody. Nanobodies can also be derived from sharks.

The term “fusion protein,” as used herein, refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nanobody domain (e.g., a nanobody that directs the binding of the protein to a target site) and a glycosyl hydrolase enzyme. Any of the proteins provided herein may be produced by any method known in the art. For example, the proteins provided herein may be produced via recombinant protein expression and purification, which is especially suited for fusion proteins comprising a peptide linker or no linker. Methods for recombinant protein expression and purification are well known and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “HAT domain” as used herein refers to a histone acetyltransferase domain. Histone acetyltransferases are enzyme that transfer an acetyl group from acetyl-CoA to conserved lysine amino acid residues on histone proteins. OGA enzymes comprise a HAT domain and display histone acetyltransferase activity in vitro.

The terms “glycan,” “sugar,” “carbohydrate,” or “saccharide,” are used interchangeably herein and refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. An exemplary monosaccharide is O-linked N-acetylglucosamine (O-GlcNAc). Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, respectively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a R anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, trehalose, and cellobiose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein. In some embodiments, the glycan is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, and derivatives thereof (e.g., N-acetylglucosamine, N-acetylgalactosamine, etc.).

The term “glycosylation,” as used herein, is the reaction in which a glycosyl donor is attached to a functional group of a glycosyl acceptor. In some embodiments, glycosylation may refer to an enzymatic process that attaches glycans to proteins. In some embodiments, glycosylation may refer to an enzymatic process that attaches glycans to other glycans already attached to a protein. In some embodiments, glycosylation is the transfer of saccharide moieties to other molecules. In some embodiments, glycosylation refers to the modification of amino acids, such as serine and threonine, through their hydroxyl groups on proteins.

The term “glycosidic bond,” as used herein, refers to a type of covalent bond that joins a carbohydrate to another group.

A “transcription factor” is a type of protein that is involved in the process of transcribing DNA into RNA. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. These exemplary human transcription factors include, but are not limited to, AC008770.3, AC023509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMPI, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLIl, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERFI, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, ZZZ3.

“Click chemistry” is a chemical strategy introduced by Sharpless in 2001 and describes chemistry tailored to generate substances quickly and reliably by joining small units together. See, e.g., Kolb, Finn, and Sharpless, Angew Chem Int Ed 2001, 40, 2004; Evans, Australian Journal of Chemistry 2007, 60, 384. The term “click chemistry” does not refer to a specific reaction or set of reaction conditions, but instead refers to a class of reactions (e.g., coupling reactions). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; and Michael additions (e.g., maleimide addition). Examples of click chemistry reactions can be found in, e.g., Kolb, H. C.; Finn, M. G. and Sharpless, K. B. Angew. Chem. Int. Ed. 2001, 40, 2004; Kolb, H. C. and Sharpless, K. B. Drug Disc. Today 2003, 8, 112; Rostovtsev, V. V.; Green L. G.; Fokin, V. V. and Sharpless, K. B. Angew. Chem. Int. Ed. 2002, 41, 2596; Tomoe, C. W.; Christensen, C. and Meldal, M. J. Org. Chem. 2002, 67, 3057; Wang, Q. et al. J. Am. Chem. Soc. 2003, 125, 3192; Lee, L. V. et al. J. Am. Chem. Soc. 2003, 125, 9588; Lewis, W. G. et al. Angew. Chem. Int. Ed. 2002, 41, 1053; Manetsch, R. et al., J. Am. Chem. Soc. 2004, 126, 12809; Mocharla, V. P. et al. Angew. Chem. Int. Ed. 2005, 44, 116; each of which is incorporated by reference herein. In some embodiments, the click chemistry reaction involves a reaction with an alkyne moiety comprising a carbon-carbon triple bond (i.e., an alkyne handle). In some embodiments, the click chemistry reaction is a copper (I)-catalyzed azide-alkyne cycloaddition (CuAAC) reaction. A CuAAC reaction generates a 1,4-disubstituted-1,2,3-triazole product (i.e., a 5-membered heterocyclic ring). See, e.g., Hein J. E.; Fokin V. V. Chem Soc Rev, 2010, 39, 1302; which is incorporated herein by reference.

The term “sample” may be used to generally refer to an amount or portion of something (e.g., a protein). A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. The “sample” may be any biological sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments a sample comprises cells, tissue, or cellular material (e.g., material derived from cells, such as a cell lysate, or fraction thereof). A sample of a cell line comprises a limited number of cells of that cell line. In some embodiments, a sample may be obtained from an individual who has been diagnosed with or is suspected of having a disease.

The term “linker,” as used herein, refers to a bond (e.g., covalent bond), chemical group, or a molecule linking two molecules or moieties, e.g., two domains of a fusion protein, such as, for example, a nanobody domain and a glycan modifying domain (e.g., a glycan modifying enzyme). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making amino acid substitutions (mutations) are known in the art and are provided in, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single- and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The terms “condition,” “disease,” and “disorder” are used interchangeably.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “cancer” refers to a group of diseases defined by the uncontrollable proliferation of abnormal cells. Examples of cancers include, but are not limited to, adenocarcinoma; anal cancer; appendix cancer; bladder cancer; breast cancer; brain cancer; cervical cancer; colorectal cancer; connective tissue cancer; esophageal cancer; ocular cancer; gall bladder cancer; gastric cancer; germ cell cancer; head and neck cancer; throat cancer; kidney cancer; liver cancer; lung cancer; muscle cancer; leukemia; bone cancer; ovarian cancer; pancreatic cancer; prostate cancer; and thyroid cancer.

The term “diabetes” refers to diabetes mellitus, which is a group of metabolic disorders defined by prolonged periods of high blood sugar level. Diabetes may be type 1 diabetes, characterized by the failure of the pancreas to produce enough insulin. Diabetes may also be type 2 diabetes, characterized by the failure of the cells of the body to respond properly to the insulin produced by the pancreas. Diabetes may also be gestational diabetes.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the inhibitor compound are outweighed by the therapeutically beneficial effects.

As used herein, “therapeutic agent” broadly refers to all agents capable of treating a condition of interest. In one embodiment of the present invention, “therapeutic drug” may be a pharmaceutical composition comprising an effective ingredient and one or more pharmacologically acceptable carriers. A pharmaceutical composition can be manufactured, for example, by mixing an effective ingredient and the above-described carriers by any method known in the technical field of pharmaceuticals. Further, mode of usage of a therapeutic drug is not limited, as long as it is used for treatment. A therapeutic drug may be an effective ingredient alone or a mixture of an effective ingredient and any ingredient. Further, the type of the above-described carriers is not particularly limited.

“Contact,” “contacting,” and similar terms as used herein may refer to either direct or indirect contact, or both.

A “variant” of a particular polypeptide or polynucleotide has one or more additions, substitutions, and/or deletions with respect to the polypeptide or polynucleotide, which may be referred to as the “original polypeptide” or “original polynucleotide,” respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments, a fragment or variant is at least 20%, 30%, 40%, 50%, 60%, 70%, 80%, 90%, 95%, 96%, 97%, 98%, 99%, or more as long as the original polypeptide or polynucleotide.

In some embodiments a variant is a biologically active variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological structure or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments, a variant, e.g., a biologically active variant, comprises or consists of a polypeptide at least 95%, 96%, 97%, 98%, 99%, 99.5%, or 100% identical to an original polypeptide over at least 70%, 75%, 80%, 85%, 90%, 95%, 96%, 97%, 98%, 99%, or 100% of the original polypeptide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. Variants may be tested in one or more suitable assays to assess activity.

As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. In certain embodiments, an antibody is an antibody fragment. However, in some embodiments, an antibody is a Fab fragment, a F(ab′)2 fragment, a Fv fragment, or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ), or mu (μ) heavy chain. In some embodiments, the heavy chain of an antibody described herein comprises a human alpha (α), delta (Δ), epsilon (ε), gamma (γ), or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (7) heavy chain constant region, such as any known in the art. Non-limiting examples of human constant region sequences have been described in the art, e.g., see U.S. Pat. No. 5,693,780. In some embodiments, the VH domain comprises an amino acid sequence that is at least 70%, 75%, 80%, 85%, 90%, 95%, 98%, or at least 99% identical to any of the variable chain constant regions. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger et al., Proceedings of the National Academy of Sciences 1993, 90, 6444; Poljak et al., Structure 1994, 2, 1121).

### DETAILED DESCRIPTION OF CERTAIN EMBODIMENTS

The aspects described herein are not limited to specific embodiments, methods, or configurations, and as such can, of course, vary. The terminology used herein is for the purpose of describing particular aspects only and, unless specifically defined herein, is not intended to be limiting.

The present disclosure provides fusion proteins comprising a nanobody and a glycosyl hydrolase enzyme (e.g., enzyme involved in removing a glycan) or a split glycosyl hydrolase enzyme. Further provided herein are split OGA enzymes. Also provided herein are methods of deglycosylating a protein using a fusion protein as described herein. Further provided in the present disclosure are methods and uses of treating and/or diagnosing diseases using the fusion proteins described herein. Also provided herein are polynucleotides encoding the fusion proteins, vectors comprising such polynucleotides, cells comprising such polynucleotides or vectors, and kits comprising any of the fusion proteins, pharmaceutical compositions, polynucleotides, vectors, or cells disclosed herein.

The present disclosure provides fusion proteins allowing for the specific and directed modification of target proteins by removal of a glycan, thus altering the molecular structure of the target proteins. In certain embodiments, the change in molecular structure results in conformational changes. In certain embodiments, these changes in structure and conformation have implications regarding the functions and interactions of the protein. In some aspects, the removal of a glycan will impact the ability of the protein to form aggregates, which are often correlated in diseases.

### Fusion Proteins

In certain embodiments, the fusion protein comprises a nanobody and a split glycosyl hydrolase. In some embodiments, the split glycosyl hydrolase comprises more than one piece. In some embodiments, the nanobody and the split glycosyl hydrolase are connected via a linker consisting of a short peptide sequence. A glycosyl hydrolase is a type of enzyme that catalyzes the hydrolysis of a glycosidic bond by excising a glycan to a glycosyl acceptor. In some embodiments, only a fragment of a glycosyl hydrolase is used in the fusion protein. In some embodiments, a variant of a glycosyl hydrolase is used in the fusion protein. In some embodiments, the fusion protein comprises only certain domains of a glycosyl hydrolase. In some embodiments, the fusion protein comprises the catalytic domain of the glycosyl hydrolase. In certain embodiments, the fusion protein comprises the stalk domain of the glycosyl hydrolase. In certain embodiments, the glycosyl hydrolase is split and comprises a first piece comprising a catalytic domain and a second piece comprising a stalk domain. In some embodiments, the catalytic domain comprises a truncated catalytic domain. In some embodiments, the stalk domain comprises a truncated stalk domain. In certain embodiments, the catalytic domain comprises a truncated catalytic domain, and the stalk domain comprises a truncated stalk domain. In some embodiments, the catalytic domain comprises amino acid residues 1-400 of SEQ ID NO: 1. In certain embodiments, the stalk domain comprises amino acid residues 544-706 of SEQ ID NO: 1. In some embodiments, the fusion protein comprises a catalytic domain comprising amino acid residues 1-400 of SEQ ID NO: 1 and a stalk domain comprising amino acid residues 544-706 of SEQ ID NO: 1. In some embodiments, the nanobody is fused to the N terminus of a piece that comprises a stalk domain. In certain embodiments, the glycosyl hydrolase is O-GlcNAcase (OGA). Exemplary glycosyl hydrolases include α-amylase, β-amylase, glucan 1,4-α-glucosidase, cellulase, endo-1,3(4)-β-glucanase, inulinase, endo-1,4-β-xylanase, oligo-1,6-glucosidase, dextranase, chitinase, polygalacturonase, lysozyme, exo-α-sialidase, α-glucosidase, β-glucosidase, α-galactosidase, β-galactosidase, α-mannosidase, β-mannosidase, β-fructofuranosidase, α,α-trehalase, β-glucuronidase, endo-1,3-β-xylanase, amylo-1,6-glucosidase, hyaluronoglucosaminidase, hyaluronoglucuronidase, xylan 1,4-β-xylosidase, β-D-fucosidase, glucan endo-1,3-β-D-glucosidase, α-L-rhamnosidase, pullulanase, GDP-glucosidase, β-L-rhamnosidase, fucoidanase, glucosylceramidase, galactosylceramidase, galactosylgalactosylglucosylceramidase, sucrose α-glucosidase, α-N-acetylgalactosaminidase, α-N-acetylglucosaminidase, α-L-fucosidase, β-L-N-acetylhexosaminidase, β-N-acetylgalactosaminidase, cyclomaltodextrinase, non-reducing end α-L-arabinofuranosidase, glucuronosyl-disulfoglucosamine glucuronidase, isopullulanase, glucan 1,3-β-glucosidase, glucan endo-1,3-α-glucosidase, glucan 1,4-α-maltotetraohydrolase, mycodextranase, glycosylceramidase, 1,2-α-L-fucosidase, 2,6-β-fructan 6-levanbiohydrolase, levanase, quercitrinase, galacturan 1,4-α-galacturonidase, isoamylase, glucan 1,6-α-glucosidase, glucan endo-1,2-β-glucosidase, xylan 1,3-β-xylosidase, licheninase, glucan 1,4-β-glucosidase, glucan endo-1,6-β-glucosidase, L-iduronidase, mannan 1,2-(1,3)-α-mannosidase, mannan endo-1,4-β-mannosidase, fructan β-fructosidase, β-agarase, exo-poly-α-galacturonosidase, κ-carrageenase, glucan 1,3-α-glucosidase, 6-phospho-β-galactosidase, 6-phospho-β-glucosidase, capsular-polysaccharide endo-1,3-α-galactosidase, non-reducing end β-L-arabinopyranosidase, arabinogalactan endo-β-1,4-galactanase, cellulose 1,4-β-cellobiosidase (non-reducing end), peptidoglycan β-N-acetylmuramidase, α,α-phosphotrehalase, glucan 1,6-α-isomaltosidase, dextran 1,6-α-isomaltotriosidase, mannosyl-glycoprotein endo-β-N-acetylglucosaminidase, endo-α-N-acetylgalactosaminidase, glucan 1,4-α-maltohexaosidase, arabinan endo-1,5-α-L-arabinanase, mannan 1,4-mannobiosidase, mannan endo-1,6-α-mannosidase, blood-group-substance endo-1,4-β-galactosidase, keratan-sulfate endo-1,4-β-galactosidase, steryl-β-glucosidase, strictosidine β-glucosidase, mannosyl-oligosaccharide glucosidase, protein-glucosylgalactosylhydroxylysine glucosidase, lactase, endogalactosaminidase, 1,3-α-L-fucosidase, 2-deoxyglucosidase, mannosyl-oligosaccharide 1,2-α-mannosidase, mannosyl-oligosaccharide 1,3-1,6-α-mannosidase, branched-dextran exo-1,2-α-glucosidase, glucan 1,4-α-maltotriohydrolase, amygdalin β-glucosidase, prunasin β-glucosidase, vicianin β-glucosidase, oligoxyloglucan β-glycosidase, polymannuronate hydrolase, maltose-6′-phosphate glucosidase, endoglycosylceramidase, 3-deoxy-2-octulosonidase, raucaffricine β-glucosidase, coniferin 3-glucosidase, 1,6-α-L-fucosidase, glycyrrhizinate β-glucuronidase, endo-α-sialidase, glycoprotein endo-α-1,2-mannosidase, xylan α-1,2-glucuronosidase, chitosanase, glucan 1,4-α-maltohydrolase, difructose-anhydride synthase, neopullulanase, glucuronoarabinoxylan endo-1,4-β-xylanase, mannan exo-1,2-1,6-α-mannosidase, α-glucuronidase, lacto-N-biosidase, 4-α-D-1(1→4)-α-D-glucanoItrehalose trehalohydrolase, limit dextrinase, poly(ADP-ribose) glycohydrolase, 3-deoxyoctulosonase, galactan 1,3-β-galactosidase, β-galactofuranosidase, thioglucosidase, β-primeverosidase, oligoxyloglucan reducing-end-specific cellobiohydrolase, xyloglucan-specific endo-β-1,4-glucanase, mannosylglycoprotein endo-β-mannosidase, fructan β-(2,1)-fructosidase, fructan β-(2,6)-fructosidase, xyloglucan-specific exo-β-1,4-glucanase, oligosaccharide reducing-end xylanase, 1-carrageenase, α-agarase, α-neoagaro-oligosaccharide hydrolase, β-apiosyl-β-glucosidase, λ-carrageenase, 1,6-α-D-mannosidase, galactan endo-1,6-β-galactosidase, exo-1,4-β-D-glucosaminidase, heparanase, baicalin-β-D-glucuronidase, hesperidin 6-O-α-L-rhamnosyl-β-D-glucosidase, protein O-GlcNAcase, mannosylglycerate hydrolase, rhamnogalacturonan hydrolase, unsaturated rhamnogalacturonyl hydrolase, rhamnogalacturonan galacturonohydrolase, rhamnogalacturonan rhamnohydrolase, β-D-glucopyranosyl abscisate β-glucosidase, cellulose 1,4-β-cellobiosidase (reducing end), α-D-xyloside xylohydrolase, β-porphyranase, gellan tetrasaccharide unsaturated glucuronyl hydrolase, unsaturated chondroitin disaccharide hydrolase, galactan endo-β-1,3-galactanase, 4-hydroxy-7-methoxy-3-oxo-3,4-dihydro-2H-1,4-benzoxazin-2-yl glucoside β-D-glucosidase, UDP-N-acetylglucosamine 2-epimerase (hydrolysing), UDP-N,N′-diacetylbacillosamine 2-epimerase (hydrolysing), non-reducing end β-L-arabinofuranosidase, protodioscin 26-O-β-D-glucosidase, (Ara-f)3-Hyp β-L-arabinobiosidase, avenacosidase, dioscin glycosidase (diosgenin-forming), dioscin glycosidase (3-O-β-D-Glc-diosgenin-forming), ginsenosidase type III, ginsenoside Rb1 β-glucosidase, ginsenosidase type I, ginsenosidase type IV, 20-β-multi-glycoside ginsenosidase, limit dextrin α-1,6-maltotetraose-hydrolase, β-1,2-mannosidase, α-mannan endo-1,2-α-mannanase, sulfoquinovosidase, exo-chitinase (non-reducing end), exo-chitinase (reducing end), endo-chitodextinase, carboxymethylcellulase, 1,3-α-isomaltosidase, isomaltose glucohydrolase, oleuropein β-glucosidase, and mannosyl-oligosaccharide α-1,3-glucosidase. In some embodiments the glycosyl hydrolase is selected from the group consisting of purine nucleosidase, inosine nucleosidase, uridine nucleosidase, AMP nucleosidase, NAD⁺ glycohydrolase, ADP-ribosyl cyclase/cyclic ADP-ribose hydrolase, adenosine nucleosidase, ribosylpyrimidine nucleosidase, adenosylhomocysteine nucleosidase, pyrimidine-5′-nucleotide nucleosidase, β-aspartyl-N-acetylglucosaminidase, inosinate nucleosidase, 1-methyladenosine nucleosidase, NMN nucleosidase, DNA-deoxyinosine glycosylase, methylthioadenosine nucleosidase, deoxyribodipyrimidine endonucleosidase, ADP-ribosylarginine hydrolase, DNA-3-methyladenine glycosylase I, DNA-3-methyladenine glycosylase II, rRNA N-glycosylase, DNA-formamidopyrimidine glycosylase, ADP-ribosyl-[dinitrogen reductase] hydrolase, N-methyl nucleosidase, futalosine hydrolase, uracil-DNA glycosylase, double-stranded uracil-DNA glycosylase, thymine-DNA glycosylase, aminodeoxyfutalosine nucleosidase, and adenine glycosylase.

In some embodiments, the nanobody portion of the fusion protein selectively binds a target. In certain embodiments, the nanobody binds a cell surface protein. In certain embodiments, the nanobody binds a target selected from the group consisting of extracellular proteins, membrane proteins, nuclear proteins, cytosolic proteins, and mitochondrial proteins. In some embodiments, the nanobody binds a target selected from the group consisting of transcription factors and nucleoporins. In some embodiments, the nanobody binds a green fluorescent protein (GFP). In certain embodiments, the nanobody binds a ubiquitin-conjugating enzyme. In some embodiments, the enzyme is UBC6e. In certain embodiments, the nanobody binds a target selected from the group consisting of c-JUN, JUNB, Sp1, and Nup62.

In some embodiments, the nanobody binds a specific peptide tag or epitope. In some embodiments, the peptide tag is a 3, 4, 5, 6, 7, 8, 9, or 10 amino acid tag. In certain embodiments, the specific peptide tag is a four-amino acid tag. In some embodiments, the four-amino acid tag is EPEA. In some embodiments, the nanobody binds the four-amino acid EPEA tag (nEPEA). In certain embodiments, the nanobody binds a Spot-tag.

### Linkers

In certain embodiments, the nanobody is fused to the glycosyl hydrolase enzyme via a linker. In certain embodiments, linkers may be used to link any of the proteins or protein domains described herein. The linker may be as simple as a covalent bond, or it may be a polymeric linker many atoms in length. In certain embodiments, the linker is a polypeptide or based on amino acids. In some embodiments, the linker is a short peptide sequence. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In certain embodiments, the linker comprises an aryl or heteroaryl moiety. In other embodiments, the linker comprises a polyethylene glycol moiety (PEG). In other embodiments, the linker comprises amino acids. In certain embodiments, the linker comprises a peptide.

The linker may include functionalized moieties to facilitate attachment of a nucleophile (e.g., thiol, amino) from the nanobody or enzyme to the linker. Any electrophile may be used as part of the linker. Exemplary electrophiles include, but are not limited to, activated esters, activated amides, Michael acceptors, alkyl halides, aryl halides, acyl halides, and isothiocyanates.

### Pharmaceutical Compositions, Polynucleotides, Vectors, and Cells

In some embodiments, the present disclosure provides a pharmaceutical composition comprising any of the fusion proteins disclosed herein. In certain embodiments, the pharmaceutical composition comprises a pharmaceutically acceptable excipient.

In some embodiments, the present disclosure provides a polynucleotide encoding a fusion protein. In some embodiments, the present disclosure provides a vector comprising the polynucleotide encoding a fusion protein described herein.

In some embodiments, the present disclosure provides a cell comprising a fusion protein. In some embodiments, the present disclosure provides a cell comprising the nucleic acid molecule encoding a fusion protein.

### Methods of Using Fusion Proteins

The present disclosure provides methods for removing a glycan from, or deglycosylating, a protein and use thereof in treating or preventing diseases or disorders (e.g., neurodegenerative diseases (Parkinson's disease, Huntington's disease, Alzheimer's disease, dementia, multiple system atrophy), cancer, and diabetes. Also provided herein is the use of fusion proteins for studying the effects of glycosylation on protein function in a cell.

In some embodiments, the present disclosure provides methods of removing a sugar from, or deglycosylating, a protein. In some embodiments, the method of removing a sugar from a protein comprises contacting a target protein containing a sugar moiety with a fusion protein, thereby removing a sugar moiety from the target protein. In some embodiments, the method of removing a sugar moiety from a protein comprises contacting a protein containing an O-linked N-acetyl glucosamine with a fusion protein described herein, thereby removing an O-linked N-acetyl glucosamine. In certain embodiments, O-linked N-acetyl glucosamine is removed from a serine or threonine residue of the protein. In some embodiments, the target protein is a transcription factor or a nucleoporin.

In certain embodiments, the target protein is a transcription factor selected from the group consisting of AC008770.3, AC023509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMPI, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLIl, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERFI, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, ZZZ3.

In certain embodiments, the target protein is a transcription factor selected from the group consisting of Sp1, JunB, and c-Jun. In some embodiments, the target protein is a nucleoporin. In certain embodiments, the nucleoporin is Nup62.

Further provided in the present disclosure are methods for studying the effects of glycosylation on protein function in a cell using any of the fusion proteins described herein. Additionally, further provided in the present disclosure are methods of treating diseases. In some embodiments, the present disclosure provides methods of treating a disease, the method comprising administering a fusion protein to a subject in need thereof.

In some embodiments, the present disclosure provides methods of treating a subject suffering from or susceptible to a neurodegenerative disease, the method comprising administering an effective amount of the fusion protein. In certain embodiments, the neurodegenerative disease is selected from the group consisting of Parkinson's disease, Huntington's disease, Alzheimer's disease, dementia, and multiple system atrophy. In some embodiments, the neurodegenerative disease is Parkinson's disease. In some embodiments, the neurodegenerative disease is Huntington's disease.

In some embodiments, the present disclosure provides methods of treating a subject suffering from or susceptible to cancer, the method comprising administering an effective amount of the fusion protein. In certain embodiments, the cancer is selected from the group consisting of bladder cancer, breast cancer, colorectal cancer, kidney cancer, lung cancer, lymphoma, melanoma, oral cancer, pancreatic cancer, prostate cancer, thyroid cancer, and uterine cancer.

In some embodiments, the present disclosure provides methods of treating a subject suffering from or susceptible to diabetes, the method comprising administering an effective amount of the fusion protein.

### Kits

In some embodiments, the present disclosure provides kits. In certain embodiments, the kit comprises any of the fusion protein disclosed herein. In some embodiments, the kit comprises any of the pharmaceutical composition disclosed herein. In some embodiments, the kit comprises a polynucleotide encoding any of the fusion proteins disclosed herein. In certain embodiments, the kit comprises a vector comprising any of said polynucleotides. In some embodiments, the kit comprises a cell comprising any of the fusion proteins or polynucleotides disclosed herein.

The kits described herein may include one or more containers housing components for performing the methods described herein and optionally instructions for uses. Any of the kit described herein may further comprise components needed for performing the methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. As used herein, “promoted” includes all methods of doing business including methods of education, scientific inquiry, academic research, and any advertising or other promotional activity including written, oral and electronic communication of any form, associated with the disclosure. Additionally, the kits may include other components depending on the specific application, as described herein.

The kits may contain any one or more of the components described herein in one or more containers. The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may also include other components, depending on the specific application, for example, containers, cell media, salts, buffers, reagents, etc.

### EXAMPLES

In order that the present disclosure may be more fully understood, the following examples are set forth. The examples described in this application are offered to illustrate the fusion proteins, compositions, kits, uses, and methods provided herein and are not to be construed in any way as limiting their scope.

### General Methods

Protein quantification by bicinchoninic acid assay was measured on a multimode microplate reader FilterMax F3 (Molecular Devices LLC, Sunnyvale, CA). Sonication on cell or protein pellets was performed using a Branson Ultrasonic Probe Sonicator (model 250). Fluorescence and chemiluminescence measurements were detected on an Azure Imager C600 (Azure Biosystems, Inc., Dublin, CA). All proteomics experiments were conducted on a Thermo Scientific EASY-nLC 1000 system connected in line to an Orbitrap Fusion Tribrid (ThermoFisher) within the Mass Spectrometry and Proteomics Resource Laboratory at Harvard University. Confocal fluorescence microscopy was performed using an Olympus confocal laser scanning microscope (FV3000).

### Cell Culture and Transfection

HEK 293T cells were cultured in Dulbecco's Modified Eagle Medium (DMEM, Cat #11995073) supplemented with penicillin (50 μg/mL) and streptomycin (50 μg/mL) along with 10% (v/v) FBS. Transfections of all plasmids in this study were performed using TransIT-PRO® (Mirus Bio, Cat #MIR 5740) according to the manufacturer's instructions.

### Plasmids and Subcloning

Glycoproteins Nup62, Sp1, JunB, c-Jun were subcloned into pcDNA3.1 vector with a GFP and a Flag-tag or a BC2 tag in the N-terminus and an EPEA tag in the C terminus respectively, unless otherwise noted. For all split OGA constructs with or without the fusion of nanobody GFP, N fragments were tagged with myc tag and C fragments were tagged with HA tag respectively. Human SP1 cDNA ORF plasmid (Cat #HG12024-G), human c-Fos cDNA ORF plasmid, and human OGT cDNA ORF cloned with a C-terminal His tag (Cat #HG11279-M) were purchased from Sino Biological. For subcloning, PCR fragments were amplified using Q5 High-Fidelity 2× Master Mix (New England BioLabs, Cat #M0492S). The vectors were double-digested and ligated to gel-purified PCR products by T4 ligation using T4 DNA ligase (New England BioLabs, Cat #M0202S) or by Gibson assembly using Gibson Assembly Master mix (New England BioLabs, Cat #E2611L). c-Fos was subcloned into a pcDNA3.1 vector with a 14-residue Ubc tag (amino acids: QADQEAKELARQIA (SEQ ID NO: 11)), Flag tag, and EPEA tag at the C terminus. All nanobody DNA fragments were synthesized by IDT. A list of genetic constructs used herein, with annotated epitope tags, vector name, etc., is provided in Table 2.

### Antibodies and Reagents

Antibodies included anti-Flag (Sigma-Aldrich, Cat #F3165), anti-OGA (Sigma-Aldrich, Cat #HPA036141), anti-DYKDDDK Tag (Cell Signaling Technology (“CST”), Cat #14793), anti-myc (CST, Cat #2276), anti-HA (CST, Cat #3724), anti-GAPDH (CST, Cat #5174), anti-OGT (CST, Cat #24083), anti-His-Tag (CST, Cat #12698), anti-OGlcNAc(RL2) (Abcam, Cat #ab2739), anti-CREB (CST, Cat #9197), anti-c-Jun (CST, Cat #60A8), anti-c-Fos (CST, Cat #2250), anti-HA-Tag (Alexa Fluor® 647 Conjugate) (CST, Cat #3444), Anti-Nup62 (BD Biosciences, Cat #610497), anti-His-Tag (Santa Cruz Biotechnology, Cat #sc-8036), anti-O-actin (Santa Cruz Biotechnology, Cat #sc-47778), anti-mouse IgG HRP (Rockland Immunochemicals, Cat #610-1302), anti-rabbit IgG HRP (Rockland Immunochemicals, Cat #611-1302), anti-mouse IgG IR 680 (LI-COR Biosciences, Cat #925-68070), anti-rabbit IgG IR 680 (LI-COR Biosciences, Cat #103011-498), Alexa Fluor™ 647 anti-rabbit IgG (Invitrogen, Cat #A21244), Alexa Fluor™ 568 anti-mouse IgG (Invitrogen, Cat #A11004). Antibody-conjugated beads for immunoprecipitation were ANTI-FLAG® M2 magnetic beads (Sigma-Aldrich, Cat #M8823) and anti-EPEA CaptureSelect™ C-tag affinity matrix (Thermo Scientific, Cat #191307005). NucBlue™ Fixed Cell Stain ReadyProbes™ reagent (Cat #R37606) was purchased from Invitrogen.

cOmplete™, EDTA-free Protease Inhibitor Cocktail (Cat #11873580001), Thiamet-G (Cat #SML0244), THPTA (Cat #762342), Cycloheximide solution (Cat #C4859), iodoacetamide (Cat #I1149), Triethylammonium bicarbonate (TEAB) buffer (Cat #T7408), BSA (Cat #A9647) and 3×FLAG® Peptide (Cat #F4799) were purchased from Sigma-Aldrich. M-PER™ mammalian protein extraction reagent (Cat #78501), Pierce C18 Tips (Cat #87784), Pierce TMT10plex™ Isobaric Mass Tag (Cat #90406), Streptavidin agarose (Cat #20353), DTT (Cat #20290) and iBlot™ Transfer Stacks (Cat #IB23001) were obtained from Thermo Scientific. Sequencing grade modified trypsin (Cat #V5111) was purchased from Promega. DBCO-PEG-5 kDa (Cat #A118) was purchased from Click Chemistry Tools. OSMI-4b, Ac45SGlcNAc and Biotin-Alkyne probe were homemade. BCA solutions (Cat #786) were purchased from G-Biosciences. Mini Bio-Spin columns (Cat #7326207) were purchased from Bio-Rad.

### Immunoprecipitation and Immunoblot Assays

After 36-48 h of transfection, cells were harvested and washed with PBS once. Cells were lysed with M-PER lysis buffer containing 1× protease inhibitor cocktail and 10 μM Thiamet-G unless otherwise noted. Protein concentrations were determined by the BCA assay kit (G-Biosciences, 786) on a multi-mode microplate reader FilterMax F3 (Molecular Devices LLC, Sunnyvale, CA).

For immunoprecipitation of proteins with the C-terminal EPEA tag, cell lysates with equal amounts of protein were diluted with PBS and incubated with C-tag affinity matrix (Thermo Scientific) for 1 h at room temperature, with end-to-end rotation, following the manufacturer's instructions. After washing three times with PBS buffer, the enriched proteins were eluted with SDS sample buffer and subjected to SDS-PAGE.

For immunoprecipitation of proteins with the Flag tag, cells were lysed in a buffer containing 50 mM Tris HCl pH 7.4, 150 mM NaCl, 1 mM EDTA, 1% Triton X-100, 5% glycerol, 1× protease inhibitor cocktail and 10 μM Thiamet-G on ice for 20 min. Cell lysates with equal amounts of protein were diluted with the lysis buffer and incubated with ANTI-FLAG® M2 magnetic beads (Sigma-Aldrich) for 2 h at 4° C. with rotation, following the manufacturer's instructions. The beads were washed by TBS buffer (50 mM Tris HCl pH 7.4, 150 mM NaCl) for three times. The enriched proteins were eluted with 3×FLAG peptide solution or SDS sample buffer and subjected to SDSPAGE.

For immunoprecipitation of proteins with anti-HA (Pierce, 88836) or anti-c-Myc (Pierce, 88842) magnetic beads, cell lysates with equal amounts of protein were diluted with 1×TBS-T buffer (25 mM Tris HCl pH 7.4, 150 mM NaCl, 0.05% Tween-20) and incubated with pre-washed magnetic beads at room temperature for 30 min with mixing, following the manufacturer's instructions. Anti-HA magnetic beads were washed three times with TBS-T buffer and once with ultrapure water. Anti-c-Myc magnetic beads were washed three times with 5×TBS-T buffer and once with ultrapure water. The enriched proteins were eluted with SDS sample buffer and subjected to SDS-PAGE.

For immunoprecipitation of proteins with His tag, cells were lysed in a buffer containing 50 mM Tris HCl pH 8.0, 150 mM NaCl, 1% Triton X-100, 5% glycerol, 1× protease inhibitor cocktail and 10 μM Thiamet-G on ice for 20 min. Cell lysates with equal amounts of protein were diluted with wash buffer (50 mM Tris HCl pH 8.0, 150 mM NaCl, 0.01% Tween-20) and incubated with pre-washed His-Tag Dynabeads (Invitrogen, 10103D) at room temperature for 20 min with mixing, following the manufacturer's instructions. After washed 4 times with wash buffer, the enriched proteins were eluted with elution buffer (300 mM Imidazole, 50 mM Tris HCl pH 8.0, 150 mM NaCl, 0.01% Tween-20) on a shaker for 10 min at room temperature.

For immunoblotting analysis, proteins were transferred to a nitrocellulose membrane using iBlot (Thermo Scientific). Membranes were blocked with Tris buffered saline containing 0.1% Tween-20 and 5% BSA and incubated with the primary antibodies (1:1000 dilution) and the secondary antibodies (1:10,000 dilution) sequentially. Immunoblot images were captured by Azure Imager C600. Analysis of immunoblots was conducted with Fiji ImageJ. All IR fluorescence western blot images are converted into grayscale images by Fiji ImageJ. The unsaturated exposure of immunoblot images was used for quantification with the appropriate loading controls as standards. Statistical analysis of the data was performed with Prism 8, using data from at least three independent experiments.

### Cycloheximide (CHX) Treatment

HEK 293T cells were transiently transfected with indicated plasmids and treated with DMSO, or Ac45SGlcNAc, or Thiamet-G at the same time if needed. 36-48 h after the transfection, cells were incubated with 50 μM CHX for up to 12 h. At the indicated time points, cells were harvested and lysed with M-PER lysis buffer. Protein expression and global O-GlcNAc level were determined by immunoblot assays. GAPDH protein level was used as the internal loading control.

### Chemoenzymatic Labeling of O-GlcNAcylated Proteins

Purification of GalT1 (Y289L) enzyme and labeling of O-GlcNAcylated proteins with GalNAz were performed according to the procedure of Hsieh-Wilson and co-workers (Thompson, J. W., Griffin, M. E. & Hsieh-Wilson, L. C. Methods for the Detection, Study, and Dynamic Profiling of O-GlcNAc Glycosylation. Methods Enzymol. 2018, 598, 101-135). Briefly, cell samples in 6-well plates or 15-cm dishes were harvested and washed by PBS once. The lysis buffer (PBS with 2% SDS) was added into cell pellets and heated for 5 min at 95° C., followed by the sonication to sheer DNA. Protein concentrations were determined by BCA assay. Cell lysates were reduced and alkylated with 25 mM DTT at 95° C. for 5 min and 50 mM iodoacetamide at room temperature for 1 h respectively. Proteins were precipitated by the methanol/chloroform solution (aqueous phase:CH₃OH:CHCl₃=4:4:1) and resuspended in 1% SDS, 20 mM HEPES (pH 7.9) buffer with a concentration of 3.75 mg/mL. For 150 μg proteins, H₂O (49 μL), 2.5×GalT labeling buffer (80 μL, final concentrations: 50 mM NaCl, 20 mM HEPES, 2% NP-40, pH 7.9), 100 mM MnCl₂ (11 μL), 500 μM UDP-GalNAz (10 μL), 2 mg/mL GalT1 (Y289L) (10 μL) were added into cell lysates orderly. The reaction was gently rotated at 4° C. for at least 20 h and the proteins were precipitated as described above. The proteins were resuspended with 1% SDS, PBS for the further click chemistry. For mass spectrometry analysis, the procedures were scaled up with the starting material of 3 mg input proteins.

Mass Shift Assay with PEG5K Labeling

For proteins in PBS containing 1% SDS, 10 mM DBCOPEG-5 kDa was added with a final concentration of 1 mM. The reaction was conducted at 95° C. for 5 min. The proteins were precipitated as previously described and resuspended in PBS containing 2% SDS. The proteins were mixed with SDS sample buffer and subjected into the immunoblot assay. The relative abundance of O-GlcNAcylated forms and unmodified form of the target protein is obtained by measuring the intensities of mass-shifted bands at higher molecular weights and the bottom band with the original molecular weight, respectively (Thompson, J. W. et al. Methods Enzymol. 2018, 598, 101-135). The ratio of abundances of O-GlcNAcylated forms versus unmodified form reflects the O-GlcNAcylation level on the protein of interest.

### CuAAC and Biotin-Immunoprecipitation

For enrichment and identification of the O-GlcNAcylated proteins, experiments were performed based on the procedure of Woo and co-workers (Woo, C. M. & Bertozzi, C. R. Isotope Targeted Glycoproteomics (IsoTaG) to Characterize Intact, Metabolically Labeled Glycopeptides from Complex Proteomes. Curr. Protoc. Chem. Biol. 2016, 8, 59-82). Briefly, the proteins in PBS containing 1% SDS were diluted with PBS and incubated with 100 μM THPTA, 0.5 mM CuSO₄, either 200 μM Biotin-PEG4-Alkyne for immunoblotting, or 200 μM Biotin-Alkyne probe for proteomics, and 2.5 mM fresh sodium ascorbate for click chemistry at 37° C. for 4 h.

For biotin-immunoprecipitation, proteins were precipitated and resuspended in 100 μL PBS containing 1% SDS. The protein solutions were diluted with PBS to lower the final concentration of SDS to 0.2% and incubated with pre-washed 40 μL streptavidin beads slurry at room temperature for 2 h with gentle rotation. Beads were washed sequentially with 0.2% SDS/PBS three times and PBS three times. Enriched proteins were eluted with SDS sample buffer and subjected to SDS-PAGE.

### Quantitative Chemical Proteomics

For quantitative proteomics, after reaction with Biotin-Alkyne probe, proteins were precipitated and resuspended in 400 μL PBS containing 2% SDS. The protein solutions were diluted with PBS to lower the final concentration of SDS into 0.2% and incubated with pre-washed 400 μL streptavidin beads slurry. The mixture was incubated at room temperature for 4 h with gentle rotation. The beads were transferred into the Bio-Spin column and washed with 1 mL 8M urea, 5 mL 0.2% SDS/PBS, 5 mL PBS and 5 mL Milli-Q water sequentially with the help of a vacuum manifold. After changing buffer with 500 μL 500 mM urea, 1 mM CaCl₂) in PBS, 2 g trypsin was added, and the resulting mixture was incubated at 37° C. for 16 h. The eluant containing trypsin digested peptides were collected as the trypsin fraction for protein identification. The peptides were desalted by C18 Tips following the manufacturer's instructions and resuspended in 20 μL 50 mM TEAB buffer. For each sample, 5 μL the corresponding amine-based TMT 10-(90406)/16-plex (A44520) reagents (Thermo Scientific, 10 μg/L, 11.9 μg/L, respectively) was added and reacted for 1 h at room temperature. The reactions were quenched with 2 μL 5% hydroxylamine solution and combined. The combined mixture was concentrated using Eppendorf Vacufuge to dryness. For glycoproteomics experiments on GFP-Sp1, the mixture was resuspended and fractionated into 6 samples with High pH Reversed-Phase Peptide Fractionation Kit (Thermo Scientific, 84868), and concentrated to dryness. All samples were stored at −20° C. until analysis.

### Mass Spectrometry Acquisition Procedures

A Thermo Scientific EASY-nLC 1000 system was coupled to a Thermo Scientific Orbitrap Fusion Tribrid with a nano-electrospray ion source. Mobile phases A and B were water with 0.1% formic acid (v/v) and acetonitrile with 0.1% formic acid (v/v), respectively. For non-fractioned peptides, peptides were separated with a linear gradient from 4 to 32% B within 140 min, followed by an increase to 50% B within 10 min and further to 98% B within 10 min, and re-equilibration. For fractionated peptides, peptides were separated with a linear gradient from 4 to 32% B within 50 min, followed by an increase to 50% B within 10 min and further to 98% B within 10 min, and re-equilibration. The instrument parameters were set as follows: survey scans of peptide precursors were performed at 120K FWHM resolution over a m/z range of 410-1800. HCD fragmentation was performed on the top 10 most abundant precursors exhibiting a charge state from 2 to 5 at a resolving power setting of 50K and fragmentation energy of 37% in the Orbitrap. CID fragmentation was applied with 35% collision energy and resulting fragments detected using the normal scan rate in the ion trap.

### Mass Spectrometry Data Analysis

The raw data was processed using Proteome Discoverer 2.4 (Thermo Fisher Scientific). For the trypsin fraction, the data were searched against the UniProt/SwissProt human (Homo sapiens) protein database (Aug. 19, 2016, 20,156 total entries) and contaminant proteins using Sequest HT algorithm. The database was adjusted by deleting 060502 (OGA) and replacing P37198 (Nup62) with GFP-Nup62 or P08047 (Sp1) with GFP-Sp1 protein sequences, respectively. Searches were performed with the following guidelines: spectra with a signal-to-noise ratio greater than 1.5; trypsin as enzyme, 2 missed cleavages; variable oxidation on methionine residues (15.995 Da); static carboxyamidomethylation of cysteine residues (57.021 Da), static TMT labeling (229.163 Da for TMT 10plex or 304.207 Da for TMT 16-plex) at lysine residues and peptide N-termini; 10 ppm mass error tolerance on precursor ions, and 0.02 Da mass error on fragment ions. Data were filtered with a peptide-to-spectrum match (PSM) of 1% FDR using Percolator. The TMT reporter ions were quantified using the Reporter Ions Quantifier without normalization. For the obtained proteome, the data was further filtered with the following guidelines: protein FDR confidence is high; unique peptides are greater than 2; master protein only; exclude all contaminant proteins.

For β-value and fold change calculations, the data was further processed using a custom algorithm as described below. Most of the empty abundances, if any, are filled in with minimum noise level. If all abundances are missing for control and treatment or the variance between existing abundances is above 30%, the PSM is removed. Applied here is a VSN normalization computed on the imputed matrix using a robust variant of the maximum-likelihood estimator for an additive-multiplicative error model and affine calibration (Huber, W. et al. Variance stabilization applied to microarray data calibration and to the quantification of differential expression. Bioinformatics. 2002, 18 Suppl 1, S96-104). The model incorporates dependence of the variance on the mean intensity and a variance stabilizing data transformation. A linear model is fitted to the expression data for control and treatment, then t-statistics are computed by empirical Bayes moderation of standard errors towards a common value.

### Immunofluorescence Microscopy

Cells were seeded on 22×22 mm glass coverslips No. 1.5 coated with poly-L-lysine (Neuvitro Corporation, Cat #H-22-1.5-pll) that had been placed in single wells of a 6-well plate for 24 h prior to transfection. Either 24-36 or 36-48 h after transfection, cells were washed with PBS twice and fixed in freshly prepared 4% paraformaldehyde in PBS for 20 min at room temperature. After washed with PBS twice, cells were permeabilized and blocked with the blocking buffer (1×PBS/5% BSA/0.3% Triton X-100) for 1 h at room temperature. The primary and secondary antibodies were diluted with the dilution buffer (1×PBS/1% BSA/0.3% Triton X-100) as the manufacturers recommended on their websites. The cells were incubated with the primary antibodies overnight at 4° C. The cells were rinsed with PBS three times, followed by 1 h incubation with the secondary antibodies (1:1000 dilution) at room temperature in the dark. The cells were washed with PBS three times and incubated with extra fluorophore conjugated primary antibodies if needed. After washing with PBS three times, NucBlue (Invitrogen) was added to stain the nuclei according to the manufacturer's instructions. Coverslips were washed with PBS and mounted in anti-fade Diamond (Life Technologies Cat #P36961). Images were collected on an Olympus confocal laser scanning microscope (FV3000). Images were exported to Fiji ImageJ for final processing and assembly.

### Luciferase Reporter Assay

For luciferase assay in the OGT inhibition experiment, HEK 293T cells co-transfected with c-Fos-Ubc-Flag-EPEA plasmid and AP-1 responsive luciferase reporter were treated with either DMSO or 25 μM OSMI-4b for 48 h before luciferase activity detection. For luciferase assay in the co-expression of nUbc-splitOGA, HEK 293T cells co-transfected with c-Fos-Ubc-Flag-EPEA plasmid and AP-1 responsive luciferase reporter with the incubation of either DMSO or 25 μM OSMI-4b were co-expressed with either nUbc-splitOGA or its inactive mutant (D174N) for 48 h before luciferase activity detection. Luciferase reporter assays were performed using Luciferase Assay System (Promega, E1500) according to manufacturer's protocols. At least three independent biological replicates were run using this assay.

### Statistical Analysis

Statistical analyses (unpaired two-tailed Student's t tests) were performed using GraphPad Prism 8. Data were derived from at least three biological replicate experiments and presented as the mean±s.d., xP<0.0332, xxP<0.0021, xxxP<0.0002, xxxxP<0.0001 and n.s., not significant.

### Example 1: Identification of the Essential Domains of OGA for Nanobody-Mediated Deglycosylation

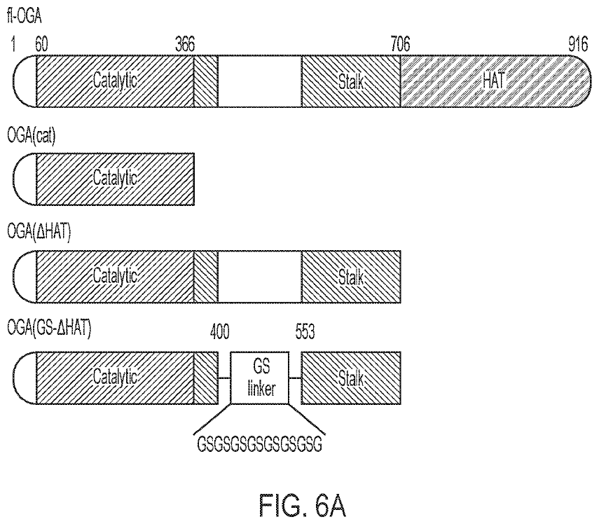

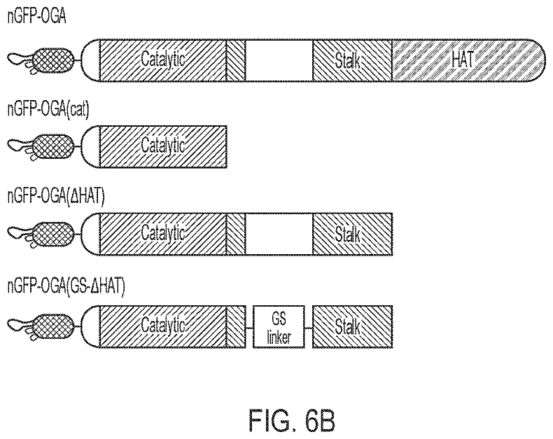

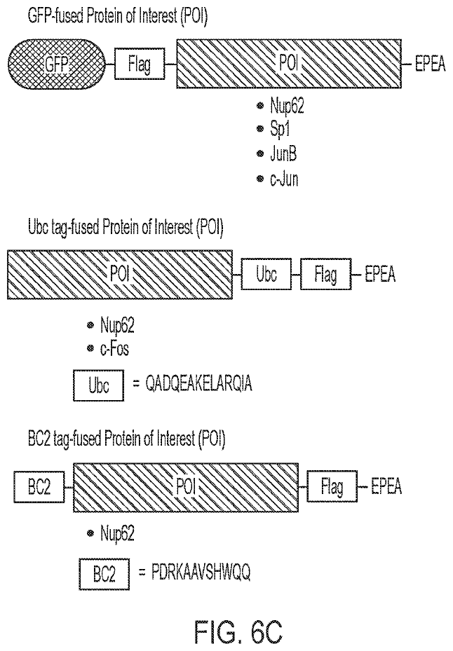

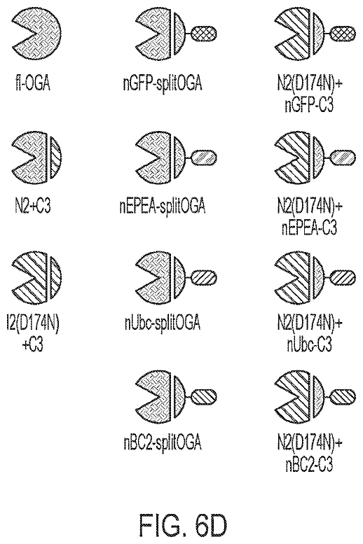

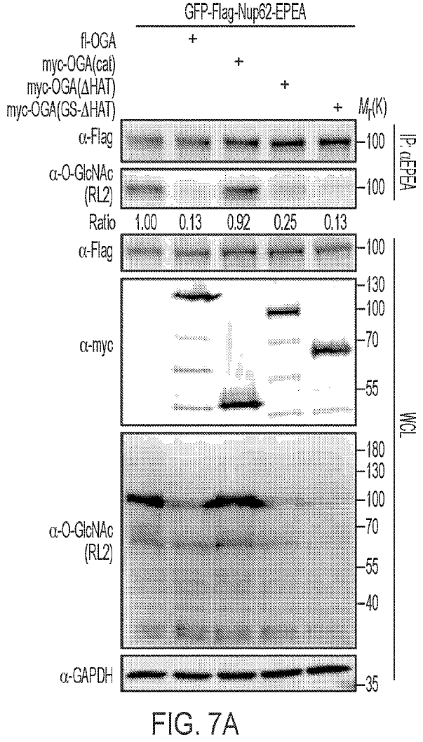

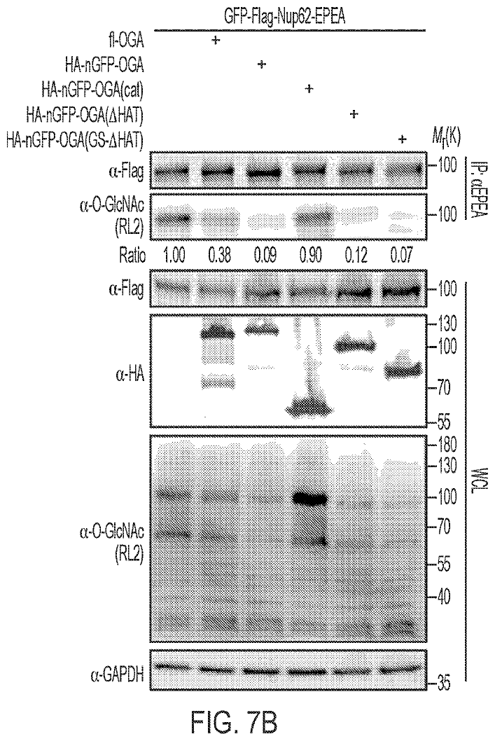

The long splice variant of human OGA is a 103-kDa hydrolase containing a catalytic domain, a stalk domain, and a pseudo-histone acetyltransferase (HAT) domain interspersed by several disordered regions (Gao, Y., Wells, L., Comer, F. I., Parker, G. J. & Hart, G. W. Dynamic O-glycosylation of nuclear and cytosolic proteins: cloning and characterization of a neutral, cytosolic beta-N-acetylglucosaminidase from human brain. J. Biol. Chem. 2001, 276, 9838-45). Three groups recently reported the crystal structure of OGA by testing several truncated constructs and screening for domain boundaries in vitro (Li, B., Li, H., Lu, L. & Jiang, J. Structures of human O-GlcNAcase and its complexes reveal a new substrate recognition mode. Nat. Struct. Mol. Biol. 2017, 24, 362-369; Roth, C. et al. Structural and functional insight into human O-GlcNAcase. Nat. Chem. Biol. 2017, 13, 610-612; Elsen, N. L. et al. Insights into activity and inhibition from the crystal structure of human O-GlcNAcase. Nat. Chem. Biol. 2017, 13, 613-615). A nominally functional OGA variant was identified taking into account its structure that targeted a substrate of interest in living cells using three constructs with or without the nanobody: (1) the catalytic domain alone [OGA(cat)], (2) a construct lacking the C-terminal HAT domain [OGA(AHAT)], and (3) a construct with a glycine-serine linker replacing a disordered region in OGA(AHAT) [OGA(GS-AHAT)] (FIGS. 6A and 6B) (Li, B., Li, H., Lu, L. & Jiang, J. Structures of human O-GlcNAcase and its complexes reveal a new substrate recognition mode. Nat. Struct. Mol. Biol. 2017, 24, 362-369). To evaluate the enzymatic activities of these constructs in living cells, Nup62 tagged with GFP and a Flag-tag at the N terminus for detection, and an EPEA-tag at the C terminus for enrichment (GFP-Nup62, FIG. 6C) were used as a target protein due to the elevated levels of O-GlcNAc across multiple glycosites (Rexach, J. E. et al. Quantification of O-glycosylation stoichiometry and dynamics using resolvable mass tags. Nat. Chem. Biol. 2010, 6, 645-51). After co-expression of GFP-Nup62 with one of the three OGA constructs in HEK 293T cells, GFP-Nup62 was immunoprecipitated and O-GlcNAcylation levels were probed with the RL2 antibody against O-GlcNAc. OGA(AHAT) and OGA(GS-AHAT), reduced O-GlcNAc on GFP-Nup62 comparable to full-length human OGA (fl-OGA), but OGA(cat) was inactive, suggesting that the catalytic domain and the stalk domain, but not the HAT domain, were required for deglycosylation of GFP-Nup62 within cells (FIG. 7A).

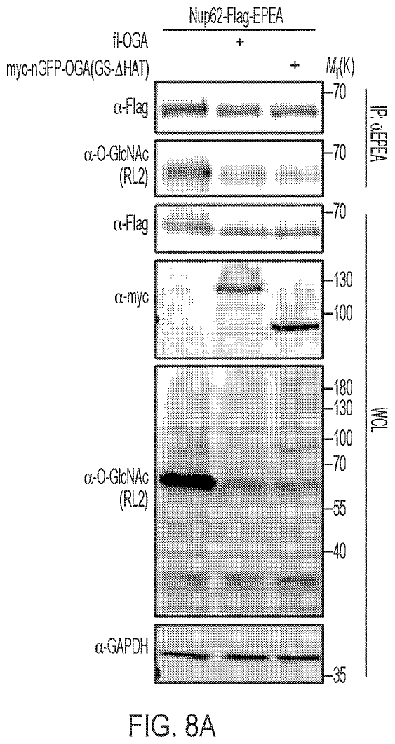

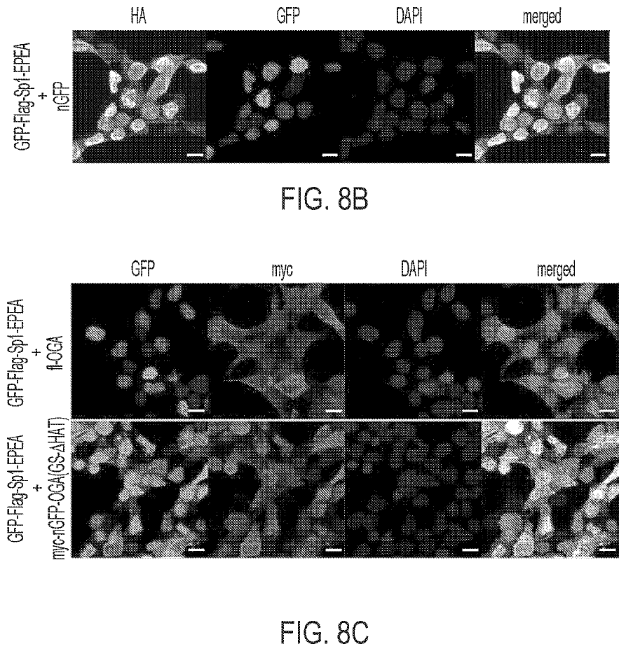

Fusions of a nanobody to OGA were evaluated to determine if they would improve O-GlcNAc removal efficiency by redirecting OGA to the target GFP-Nup62. Active OGA constructs with a nanobody against GFP (nGFP) (Kirchhofer, A. et al. Modulation of protein properties in living cells using nanobodies. Nat. Struct. Mol. Biol. 2010, 17, 133-8) at the N terminus improved deglycosylation of GFP-Nup62 (FIG. 7B). However, the nanobody-OGA fusion proteins reduced O-GlcNAc levels globally, including Nup62 lacking GFP, and affected the localization of the target protein (FIG. 8). To improve the target protein selectivity, OGA was further engineered to identify a construct capable of serving as a protein-selective O-GlcNAc eraser.

### Example 2: Optimization of a Selective Nanobody-Split OGA for Target Protein Deglycosylation

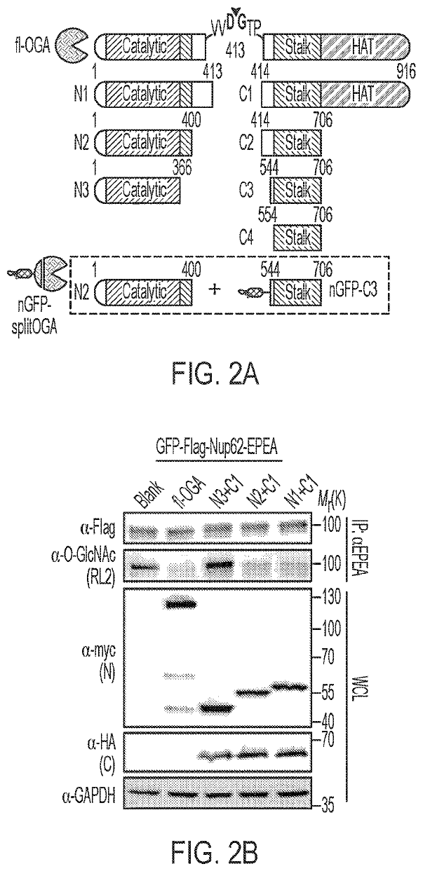

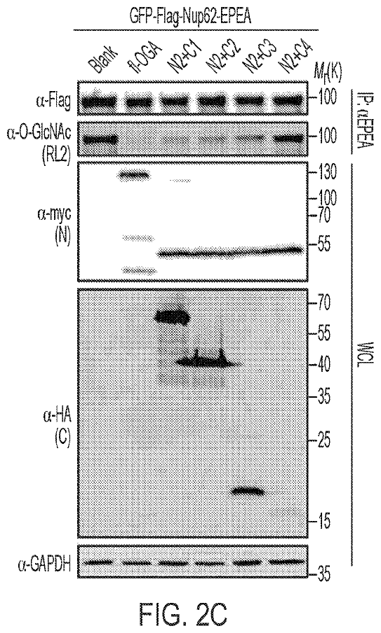

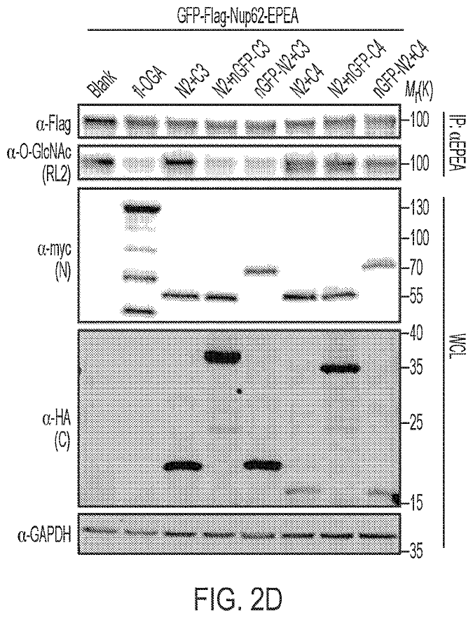

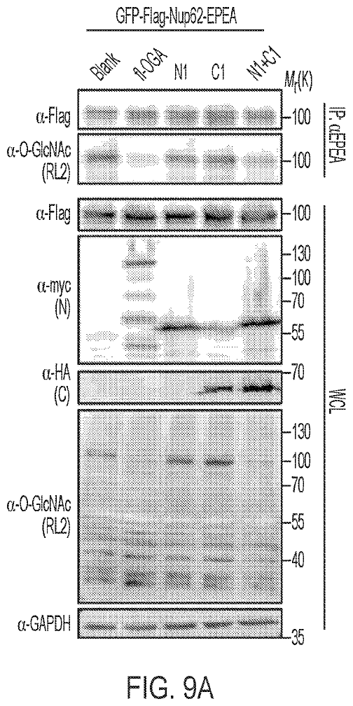

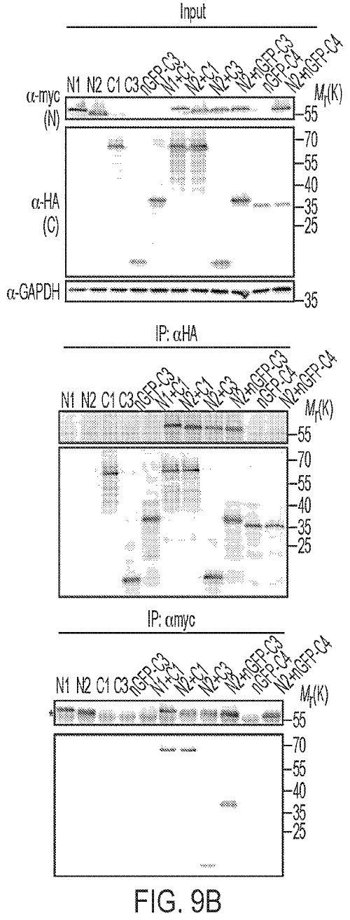

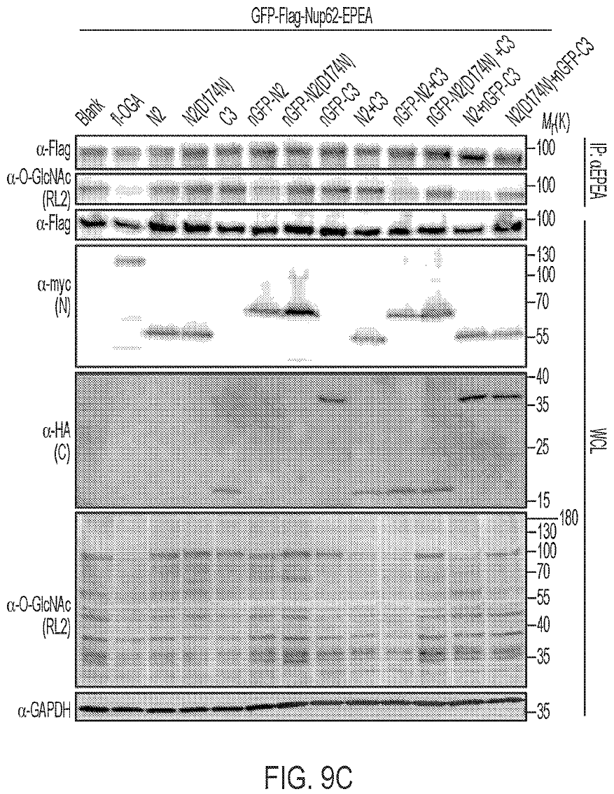

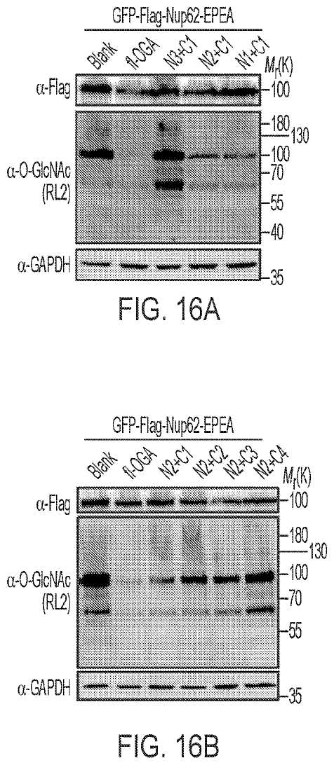

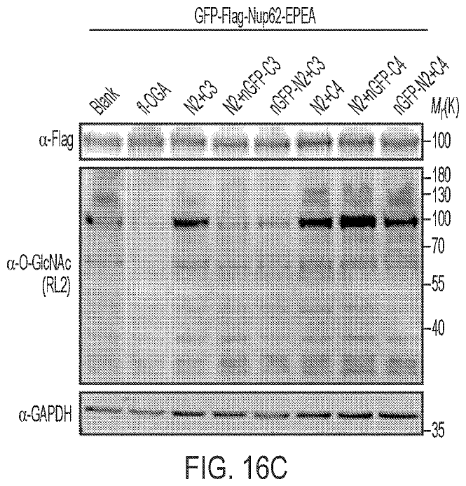

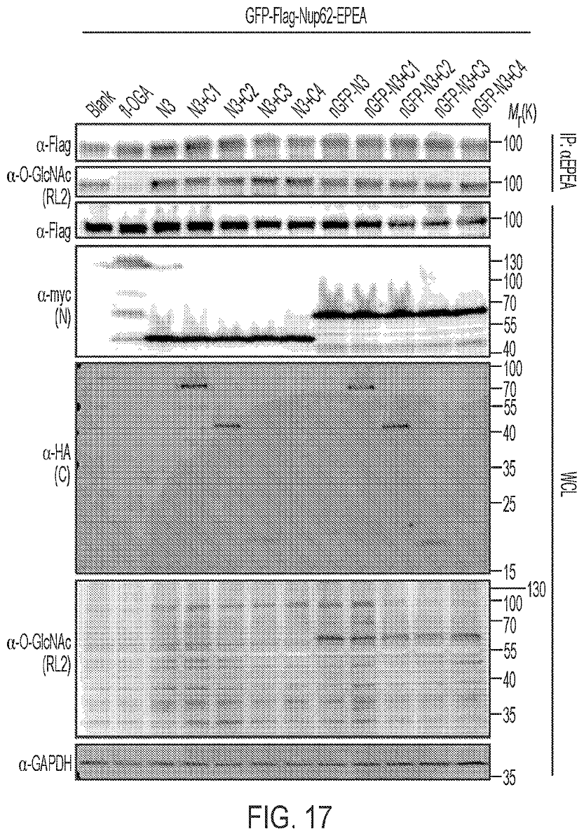

The size and activity of OGA was reduced in order to reduce the inherent activity of OGA and therefore minimizing the global perturbation of O-GlcNAc in living cells. Human OGA contains a caspase-3 cleavage site at Asp-413 that splits OGA during apoptosis into an N- and C-terminal fragment (FIG. 2A) (Butkinaree, C. et al. Characterization of beta-N-acetylglucosaminidase cleavage by caspase-3 during apoptosis. J. Biol. Chem. 2008, 283, 23557-66). The N- and C-terminal fragments can associate to reconstitute OGA enzymatic activity when co-expressed simultaneously in living cells (FIG. 9A). The association between the two fragments was lowered to reduce the off-target effect and therefore improve target protein selectivity upon installation of the nanobody. First, to finely tune the enzymatic activity of split OGA, three N fragments (N1-N3) and four C fragments (C1-C4) based on the structure of OGA were generated. These fragments iteratively reduced the size of each fragment, starting from the original cleavage site Asp-413 (FIG. 2A). The deglycosidase activity of these fragments were screened against GFP-Nup62 beginning with the truncated N1-N3 fragments paired with the original C fragment [C1, amino acid (aa) 414-916] (FIGS. 2B and 16A). Following immunoprecipitation and probing for O-GlcNAc on GFP-Nup62, it was determined that N2 was the minimal N fragment that possessed deglycosylation capability when paired with C1. When the N fragment was shortened further to N3 by complete removal of the stalk domain, no activity was seen on GFP-Nup62. Furthermore, the activity of split OGA containing N3 could not be recovered by fusing nGFP to N3 and pairing with any of the C fragments (FIG. 17). It was hypothesized that essential contacts with part of the stalk domain (aa 367-400) were critical for the biochemical activity of OGA in cells, which was consistent with in vitro observations (Elsen, N. L. et al. Insights into activity and inhibition from the crystal structure of human O-GlcNAcase. Nat. Chem. Biol. 2017, 13, 613-615).

To further optimize the activity of OGA, the C fragments in combination with N2 were screened. As before, shorter C fragments corresponded to a decrease in split OGA activity on GFP-Nup62 (FIGS. 2C and 16B). Among those combinations, the N2-C3 pair and the N2-C4 pair showed significantly reduced activity. These two less active forms of OGA were further evaluated for whether the nanobody would reinstate activity to the target GFP-Nup62. To achieve the optimal fusion strategy with nGFP, nGFP was fused to the N-terminus of either the N fragment (N2) or C fragments (C3 or C4). Indeed, fusion of nGFP to either N2 or C3 selectively restored deglycosidase activity to GFP-Nup62 (FIG. 2D). However, no activity on GFP-Nup62 was observed with the N2-C4 pair after fusion of nGFP (FIGS. 2D and 16C). Similarly, only the N2 and nGFP-fused C3 pair were reciprocally co-immunoprecipitated (FIG. 9B), which suggested that amino acids 544-553 contribute to the association of the two fragments, likely via the formation of the OGA homodimer, and therefore the deglycosylation activity of OGA in vivo (Elsen, N. L. et al., Insights into activity and inhibition from the crystal structure of human O-GlcNAcase. Nat. Chem. Biol. 2017, 13, 613-615).

The deglycosidase activity of nanobody-fused split OGA was confirmed by using a mutation of D174, one of the two catalytic aspartate residues (D174 and D175) in OGA, to asparagine (N) as an inactive negative control (FIG. 9C). Compared to the inactive control, it was found that the nGFP-fused N2 (nGFP-N2) alone minorly deglycosylated the target GFP-Nup62. It was therefore concluded that the combination of N2 with nGFP-fused C3 was the optimal combination in terms of efficiency and selectivity at removal of O-GlcNAc on GFP-Nup62, and this combination was termed nGFP-splitOGA in subsequent experiments (highlighted in the dotted rectangle, FIG. 2A).

### Example 3: nGFP-splitOGA Removes O-GlcNAc from a Series of Target Proteins

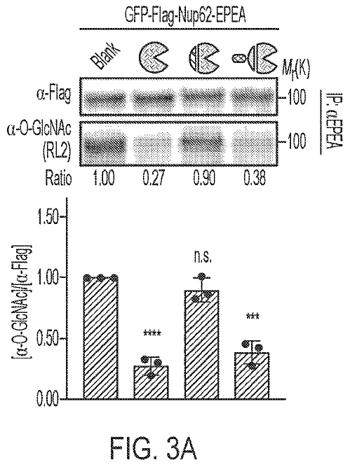

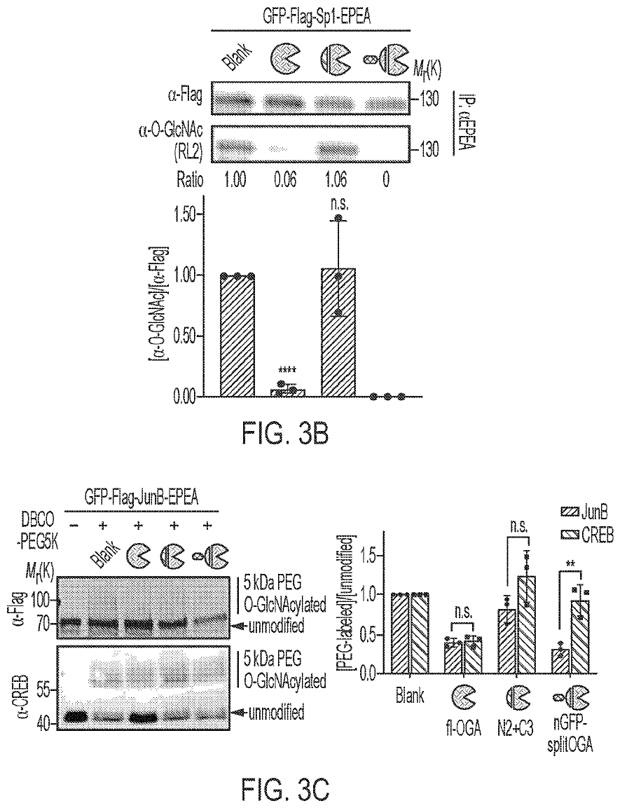

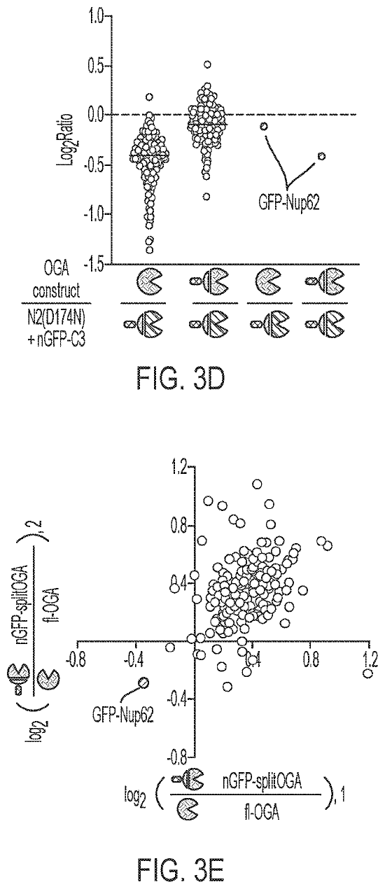

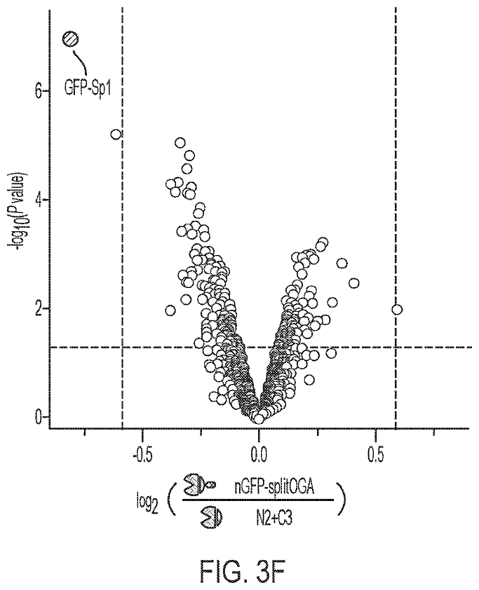

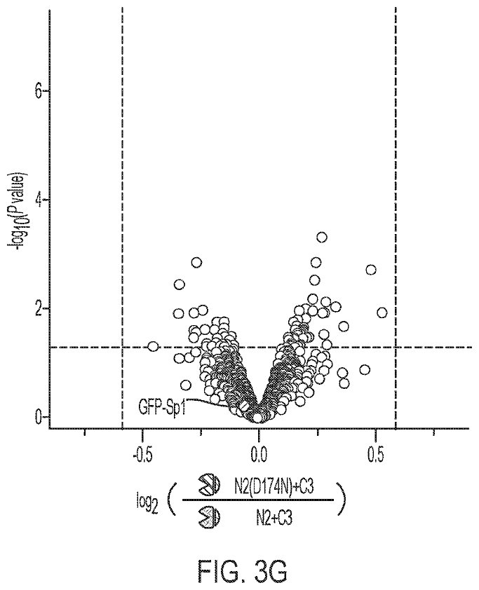

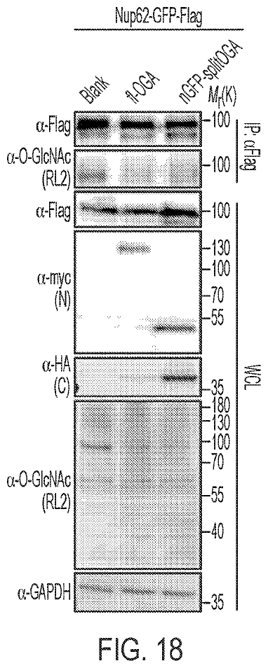

The general ability of nGFP-splitOGA to selectively remove O-GlcNAc from a range of target proteins was then evaluated. nGFP-splitOGA removed O-GlcNAc analogous to the full-length OGA (fl-OGA) from GFP-Nup62 in a nGFP-dependent manner, regardless of the GFP orientation on Nup62 (FIGS. 3A, 18, and 19A).

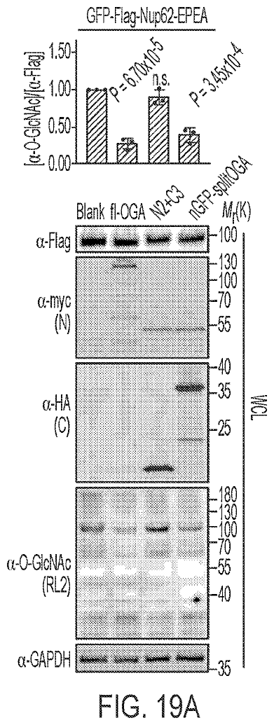

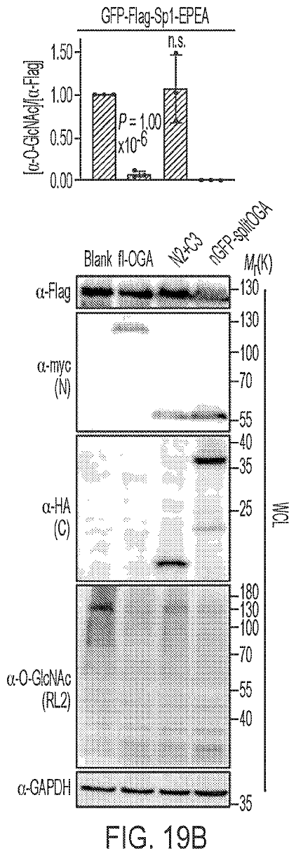

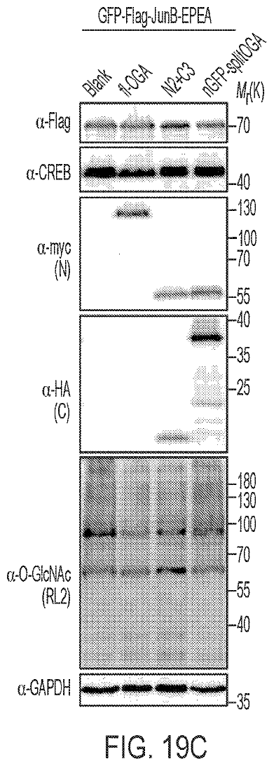

The ability to selectively deglycosylate GFP-Sp1, a transcription factor that possesses a high inherent level of O-GlcNAc, was examined (Rexach, J. E. et al. Quantification of O-glycosylation stoichiometry and dynamics using resolvable mass tags. Nat. Chem. Biol. 2010, 6, 645-51). As expected, nGFP-splitOGA selectively deglycosylated GFP-Sp1 at levels equivalent to fl-OGA (FIGS. 3B and 19B). Without nGFP, split OGA (N2+C3) alone possessed weak inherent deglycosidase activity. Furthermore, co-expression of nGFP-splitOGA with GFP-Sp1 did not alter the nuclear localization of Sp1 in HEK 293T cells despite the strong binding between nGFP and GFP, in contrast to the effects of an earlier construct, nGFP-OGA(GS-AHAT).

nGFP-splitOGA activity on GFP-JunB was examined using a mass shift assay (Rexach, J. E. et al. Quantification of O-glycosylation stoichiometry and dynamics using resolvable mass tags. Nat. Chem. Biol. 2010, 6, 645-51) to elucidate the O-GlcNAcylation level. JunB is a transcription factor with several glycosites at a relatively low occupancy (Woo, C. M. et al. Mapping and Quantification of Over 2000 O-linked Glycopeptides in Activated Human T Cells with Isotope-Targeted Glycoproteomics (Isotag). Mol. Cell. Proteomics 2018, 17, 764-775). Cell lysates co-transfected with nGFP-splitOGA and GFP-JunB were sequentially labeled with UDP-GalNAz and 5 kDa DBCO-PEG to reveal the O-GlcNAcylation level by immunoblotting. Again, deglycosylation of target protein JunB occurred selectively with nGFP-splitOGA (FIG. 3C). Notably, O-GlcNAc levels on the untargeted endogenous marker CREB24 were stable in the presence of nGFP-splitOGA, in contrast to the global reduction of O-GlcNAc produced by expression of fl-OGA (FIGS. 3C and 19C). These data pointed to the high target protein selectivity and generality of nGFP-splitOGA.

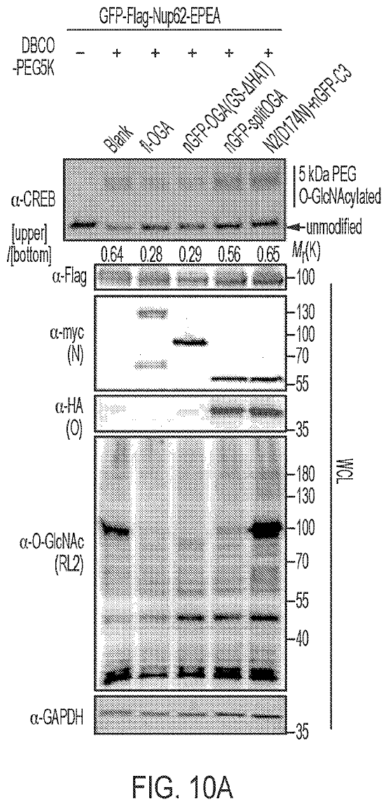

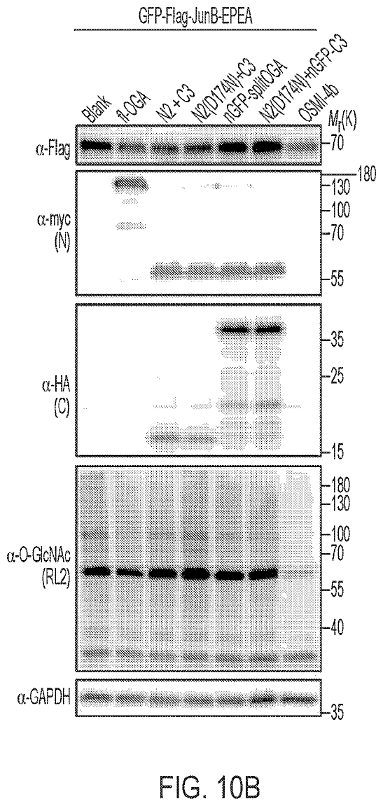

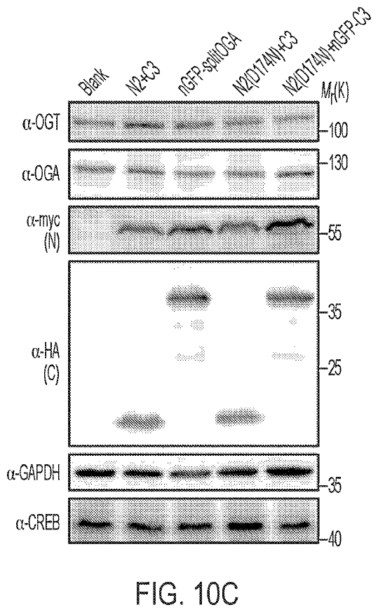

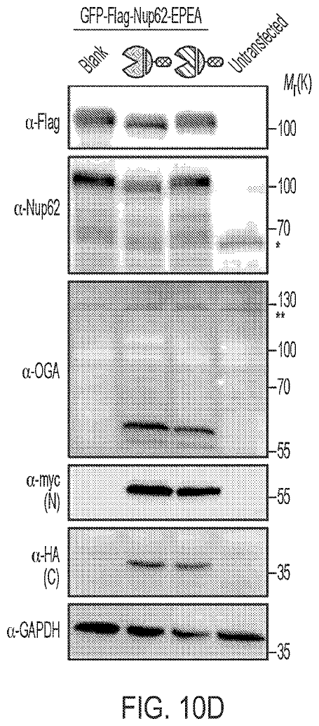

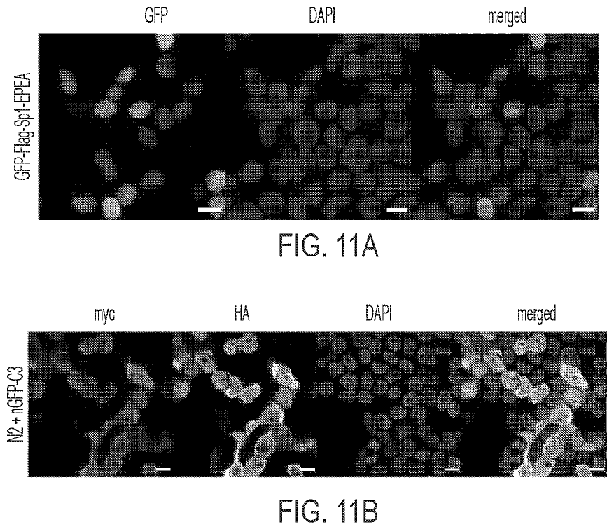

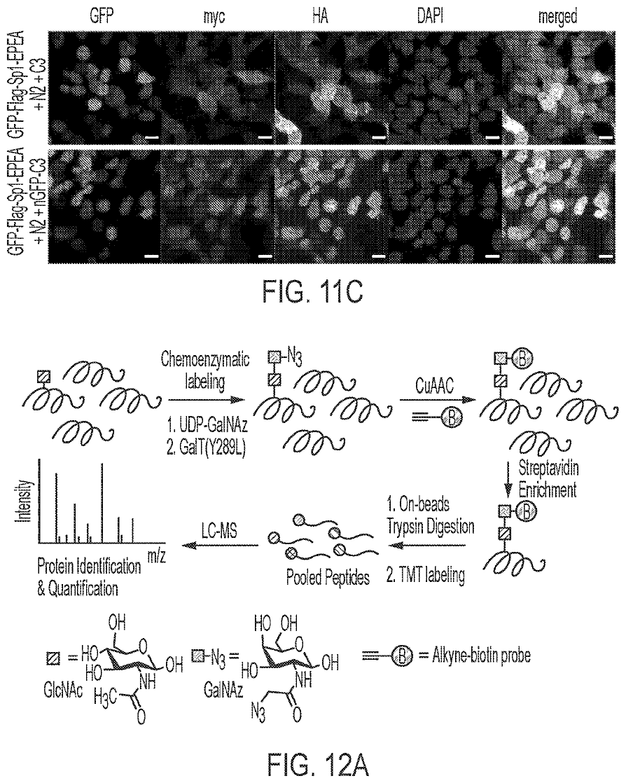

Since CREB is an endogenous marker of off-target activity of the nanobody-directed OGA constructs, the O-GlcNAcylation level on CREB following co-expression of GFP-Nup62 and several of the OGA constructs in HEK 293T cells was compared. As anticipated, nGFP-splitOGA had a limited effect on CREB O-GlcNAcylation, while both fl-OGA and nGFP-OGA(GS-AHAT) reduced O-GlcNAc on CREB and the whole proteome (FIGS. 3C and 19C). The O-GlcNAc levels on CREB were minimally perturbed by nGFP-splitOGA regardless of the target protein (FIG. 10A). Global O-GlcNAc levels also showed negligible changes on co-expression of nGFP-splitOGA and GFP-JunB, in contrast to the dramatic reduction caused by the OGT inhibitor OSMI-4b9 (FIG. 10B). Furthermore, no perturbation of endogenous OGT or OGA protein levels was observed in the presence of nGFP-splitOGA constructs, though expression levels of nGFP-splitOGA is greater than native expression of OGA (FIGS. 10C and 10D). In addition, nGFP-splitOGA did not alter the subcellular localization of the target protein in HEK 293T cells (FIG. 11). These data point to the high selectivity, orthogonality, and generality of nGFP-splitOGA in target protein deglycosylation.

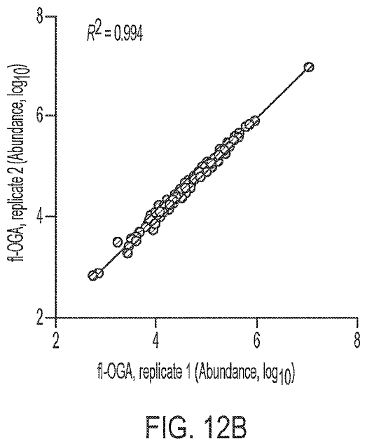

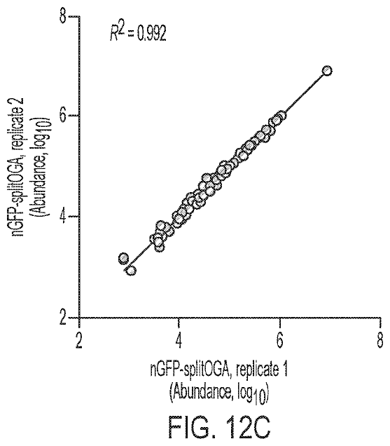

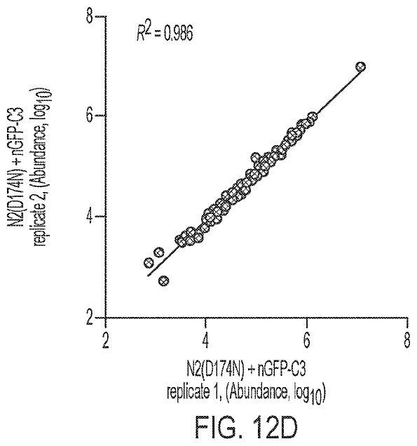

An unbiased quantitative mass spectrometry analysis of the O-GlcNAcylated proteome on expression of nGFP-splitOGA was performed to globally validate the selectivity of nGFP-splitOGA (FIG. 12A). O-GlcNAcylated proteins from HEK 293T cells co-expressing GFP-Nup62 and (1) fl-OGA, (2) nGFP-splitOGA, or (3) the inactive form of nGFP-splitOGA [N2(D174N)+nGFP-C3] were chemoenzymatically labeled, enriched, and digested for protein identification and quantification using tandem mass tags (TMT) (Ramirez, D. H. et al. Engineering a Proximity-Directed O-GlcNAc Transferase for Selective Protein O-GlcNAcylation in Cells. ACS Chem Biol 2020). Two independent biological replicates with HEK 293T cells co-expressing GFP-Nup62 and (1) full-length OGA (fl-OGA), (2) nGFP-splitOGA, or (3) the inactive form of nGFP-splitOGA [N2(D174N)+nGFP-C3], respectively, were performed with excellent correlation between the two biological replicates (FIGS. 12B-12D). Samples expressing an active OGA construct were compared to the inactive form of nGFP-splitOGA as a control. fl-OGA expression globally decreased O-GlcNAcylated proteins, while nGFP-splitOGA expression significantly reduced O-GlcNAcylated GFP-Nup62 with negligible perturbation of the O-GlcNAc proteome by comparison (FIG. 3D). The observed greater reduction on GFP-Nup62 by nGFP-splitOGA than by fl-OGA may be due to the more sensitive measurement by mass spectrometry (FIG. 3D). Direct comparison of nGFP-splitOGA to fl-OGA samples showed GFP-Nup62 as the only protein that is reproducibly deglycosylated by nGFP-splitOGA more than fl-OGA (FIG. 3E). To directly compare the nGFP-splitOGA to split OGA alone, the global O-GlcNAc proteome was subsequently quantified from four independent biological replicates with HEK 293T cells co-expressing GFP-Sp1 and (1) nGFP-splitOGA, (2) split OGA (N2+C3), or (3) the inactive form of split OGA [N2(D174N)+C3]. GFP-Sp1 displays the largest O-GlcNAc reduction with the minimal β-value on co-expression of nGFP-splitOGA in comparison to split OGA (FIG. 3F). Notably, the global O-GlcNAc proteome in the presence of split OGA shows no significant difference from that of the inactive form (FIG. 3G). Taken together, both immunoblotting analysis and quantitative proteomics demonstrated that nGFP-splitOGA is a general mechanism to selectively remove O-GlcNAc from target proteins in cells, at efficiency levels comparable to fl-OGA, while minimally affecting the broader O-GlcNAc proteome.

### Example 4: Alternative Nanobodies for Use in the Split OGA System

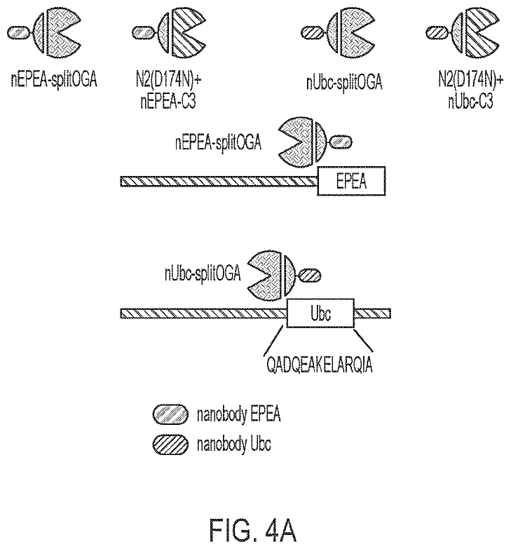

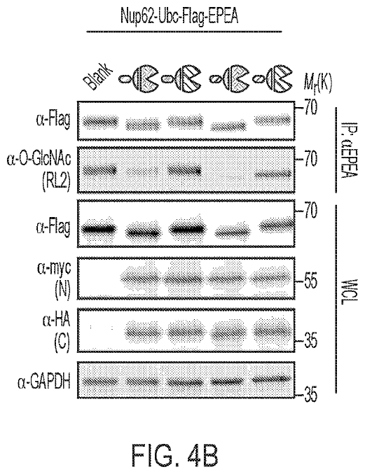

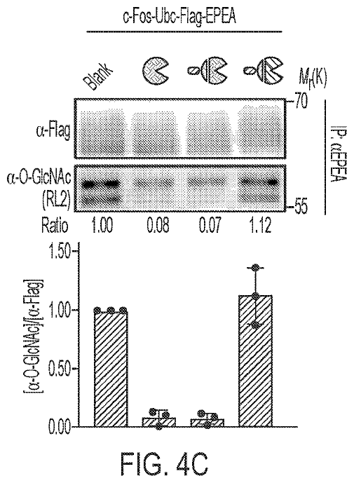

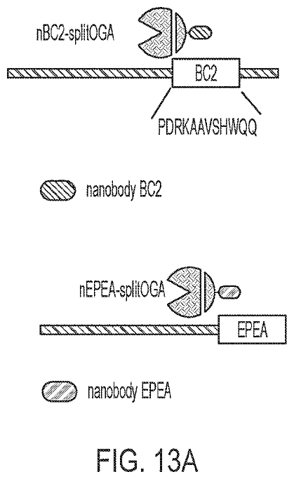

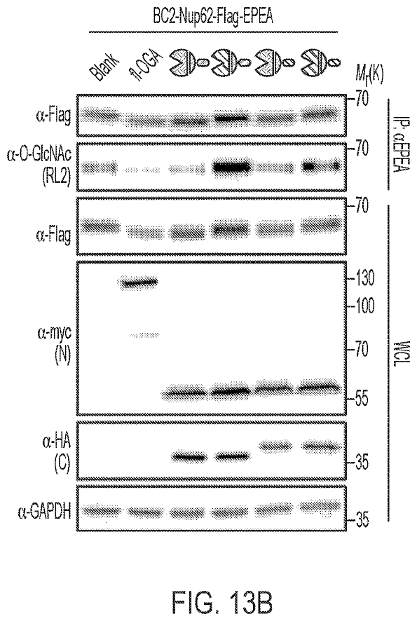

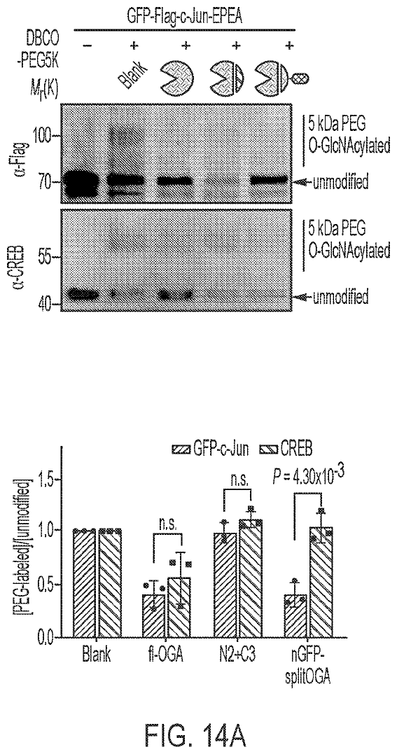

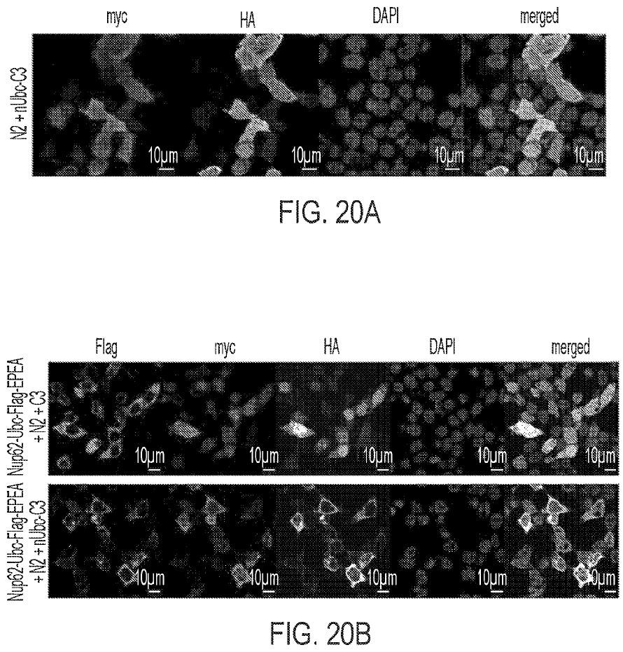

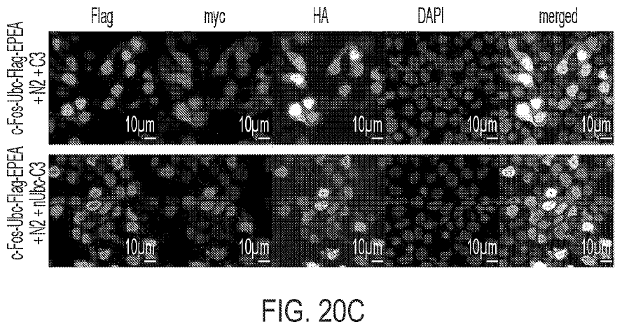

A variety of alternative nanobodies for use in the split OGA system for target protein deglycosylation were also developed. EPEA-tag is a 4-amino acid C-terminal tag that can be selectively recognized by its nanobody (named as nEPEA here) (De Genst, E. J. et al. Structure and properties of a complex of alpha-synuclein and a single-domain camelid antibody. J. Mol. Biol. 2010, 402, 326-43). Ubc-tag is a newly reported 14-amino acid peptide from the E2 ubiquitin-conjugating enzyme UBC6e, which can be recognized by its nanobody (named as nUbc hereafter) with high affinity (Ling, J. et al. A nanobody that recognizes a 14-residue peptide epitope in the E2 ubiquitin-conjugating enzyme UBC6e modulates its activity. Mol. Immunol. 2019, 114, 513-523). The model protein Nup62 was tagged with both a Ubc-tag and an EPEA-tag at the C terminus and the nGFP-split OGA system was replaced with nEPEA and nUbc respectively (FIG. 4A and FIG. 4B). Using the inactive mutant of split OGA (N2(D174N)) as the negative control, both the nEPEA-split OGA system and nUbc-split OGA system were applied on the same substrate Nup62-Ubc-Flag-EPEA. Nup62-Ubc-Flag-EPEA was enriched by anti-EPEA beads and analyzed by immunoblotting to reveal the protein level and O-GlcNAc modification level respectively. Nup62 with Ubc and EPEA tags was efficiently deglycosylated with either active nEPEA-split OGA or nUbc-split OGA, which demonstrates that multiple tag-nanobody pairs can be adapted to the generalizable split OGA system for target protein deglycosylation (FIG. 4B). The nUbc-splitOGA and the Ubc-tagged substrate were further observed to co-localize by confocal imaging (FIG. 20). Target protein deglycosylation was also effective with a third nanobody that recognizes the BC2-tag, a 12-residue peptide epitope (Traenkle, B. et al., Monitoring interactions and dynamics of endogenous beta-catenin with intracellular nanobodies in living cells. Mol. Cell. Proteomics. 2015, 14, 707-23)) (FIG. 13).

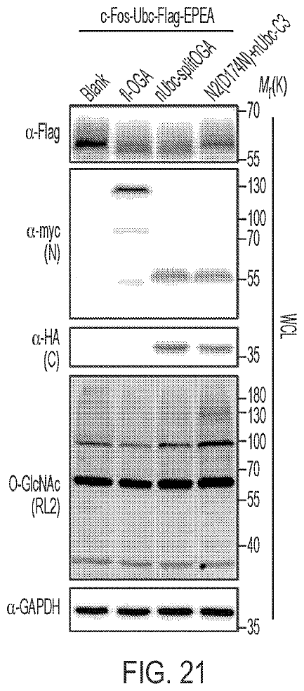

The ability of nUbc-split OGA to selectively remove an O-GlcNAc modification from Ubc-tagged c-Fos was examined. c-Fos is a core component of the AP-1 transcription factor complex, which will dimerize with c-Jun and drive downstream transcription (Hess, J., Angel, P. & Schorpp-Kistner, M. AP-1 subunits: quarrel and harmony among siblings. J. Cell Sci. 2004, 117, 5965-73). C-Fos is reported to be O-GlcNAc modified in mammalian cells (Tai, H. C., et al., Parallel identification of O-GlcNAc-modified proteins from cell lysates. J. Am. Chem. Soc. 126, 10500-1 (2004)), yet these glycosites await unambiguous assignment by MS. c-Fos-Ubc-Flag-EPEA was co-expressed with the indicated constructs and enriched by anti-EPEA beads. The O-GlcNAc modification level was evaluated by immunoblotting with an anti-O-GlcNAc antibody (FIG. 4C). Ratios were quantified from the protein level and O-GlcNAc level. Target deglycosylation of c-Fos tagged with Ubc (c-Fos-Ubc) in HEK 293T cells showed that, as expected, O-GlcNAc was selectively reduced from the target protein by nUbc-splitOGA, and not the inactive mutant [N2(D174N)+nUbc-C3], at levels equivalent to fl-OGA (FIG. 4C and FIG. 21). Taken together, these results demonstrate that multiple nanobody-tag pairs are readily adapted to split OGA for protein-selective deglycosylation.

### Example 5: Revealing the Role of O-GlcNAc on Target Proteins

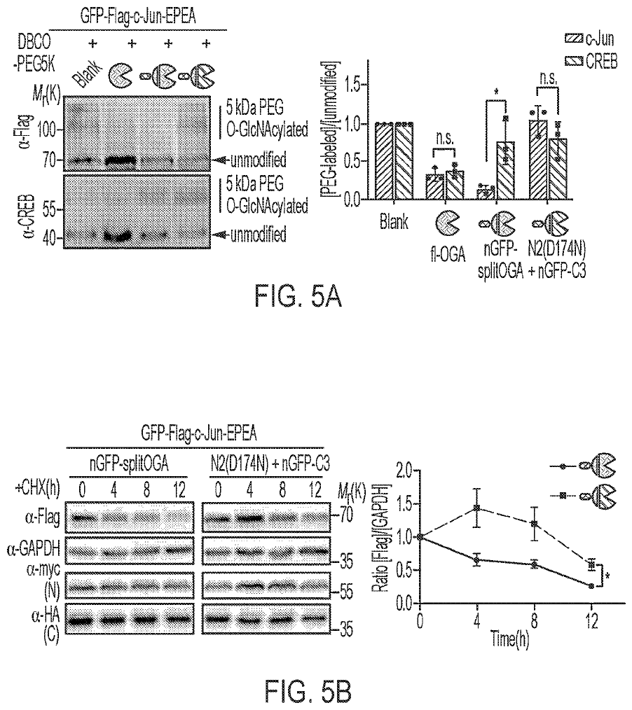

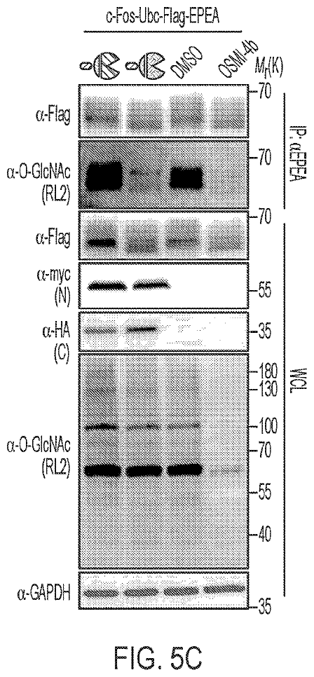

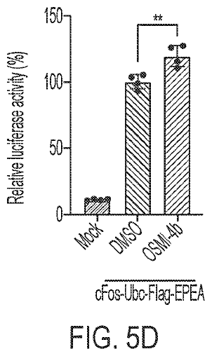

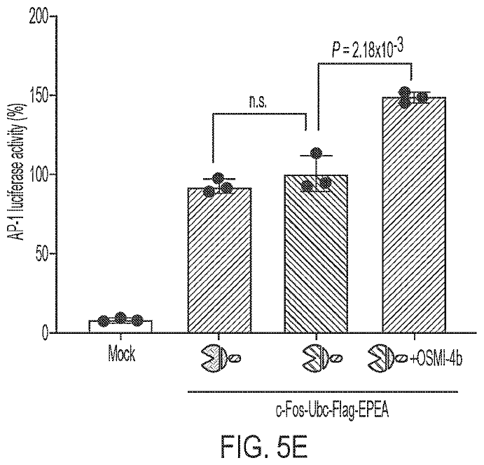

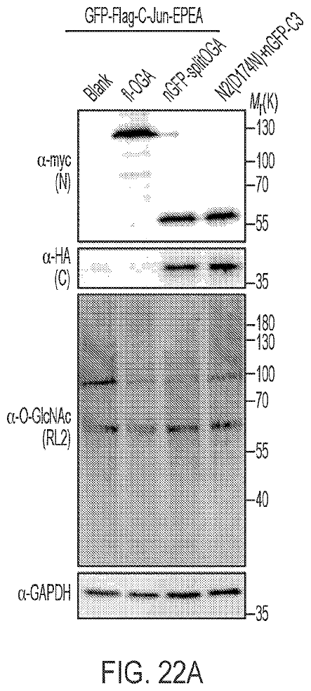

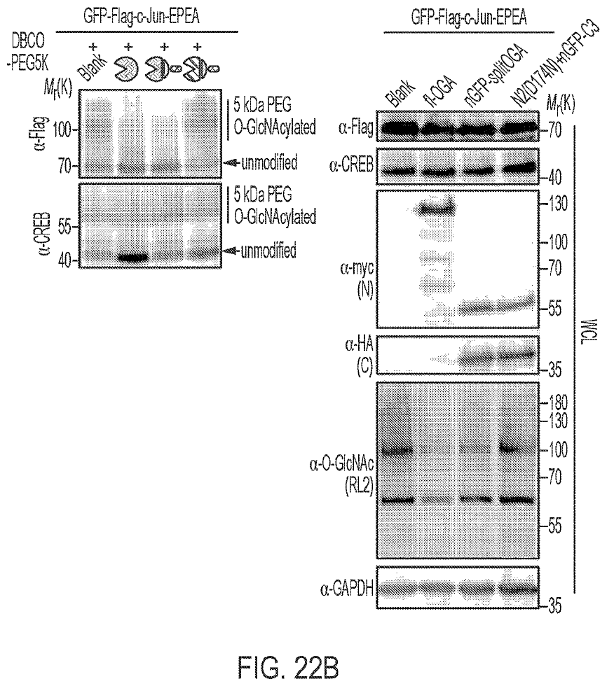

The impact of O-GlcNAc on the target protein c-Jun was used to determine if the targeted O-GlcNAc eraser would facilitate the assignment of O-GlcNAc function on a target protein. c-Jun is another core component of the AP-1 transcription factor complex that acts by dimerizing with members of the Fos family (Hess, J., Angel, P. & Schorpp-Kistner, M. AP-1 subunits: quarrel and harmony among siblings. J. Cell Sci. 2004, 117, 5965-73). c-Jun is O-GlcNAcylated at multiple sites (Kim, S., Maynard, J. C., Strickland, A., Burlingame, A. L. & Milbrandt, J. Schwann cell O-GlcNAcylation promotes peripheral nerve remyelination via attenuation of the AP-1 transcription factor JUN. Proc Natl Acad Sci USA 2018, 115, 8019-8024) and stabilized on OGT overexpression in hepatocellular carcinoma (HCC) cells (Qiao, Y. et al. High Glucose Stimulates Tumorigenesis in Hepatocellular Carcinoma Cells Through AGER-Dependent O-GlcNAcylation of c-Jun. Diabetes 2016, 65, 619-32). The direct contribution of O-GlcNAc to stabilization of c-Jun was evaluated. A mass shift assay was performed on GFP-c-Jun to validate that nGFP-splitOGA can selectively erase O-GlcNAc from O-GlcNAcylated GFP-c-Jun. Similar to GFP-JunB, GFP-c-Jun was selectively deglycosylated by nGFP-splitOGA instead of the inactive form [N2(D174N)+nGFP-C3], with minimal disruption of O-GlcNAc levels on endogenous CREB or the global O-GlcNAc proteome (FIGS. 5A, 14A-14B, and 22).

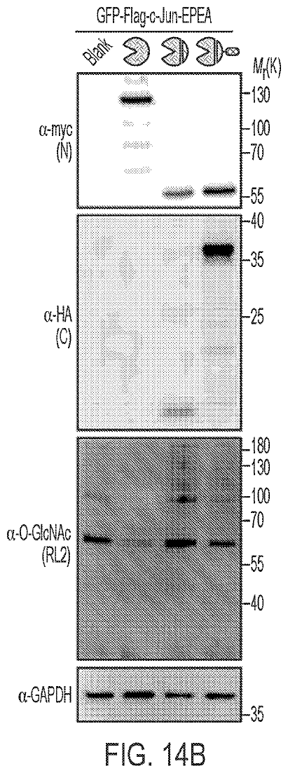

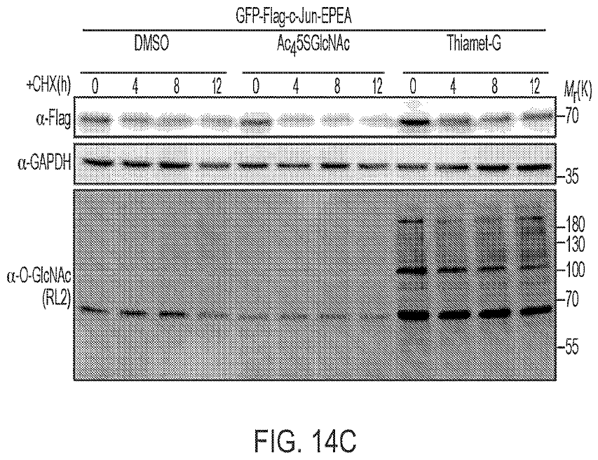

Given that O-GlcNAc may stabilize c-Jun, the turnover of GFP-c-Jun in HEK 293T cells was monitored by adding cycloheximide (CHX) to block protein synthesis. Indeed, the degradation of GFP-c-Jun was accelerated upon OGT inhibition with A_{c4}5SGlcNAc and attenuated upon OGA inhibition with Thiamet-G (FIG. 14C). However, inhibition of OGT or OGA broadly altered O-GlcNAc on a number of proteins, which may alter protein stability through other mechanisms (Zhang, F. et al. O-GlcNAc modification is an endogenous inhibitor of the proteasome. Cell. 2003, 115, 715-25). Therefore, nGFP-splitOGA was employed to directly link the O-GlcNAc modification on GFP-c-Jun to protein stability. GFP-c-Jun was co-expressed with nGFP-splitOGA or the inactive form of nGFP-splitOGA in HEK 293T cells (FIG. 4B). Upon addition of CHX, the protein level of GFP-c-Jun was monitored over time and revealed that deglycosylation of GFP-c-Jun selectively generated by nGFP-splitOGA accelerated degradation (FIG. 4B), implying that the stability of GFP-c-Jun was directly impaired by the loss of O-GlcNAc.

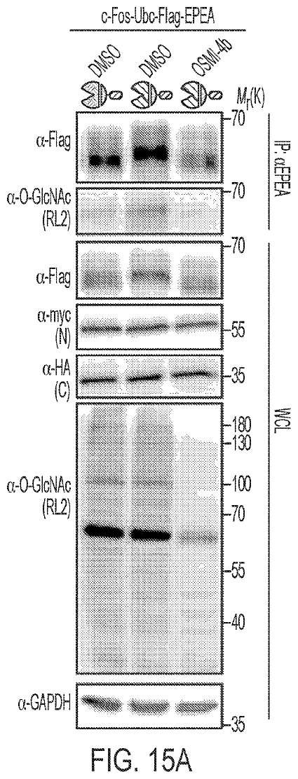

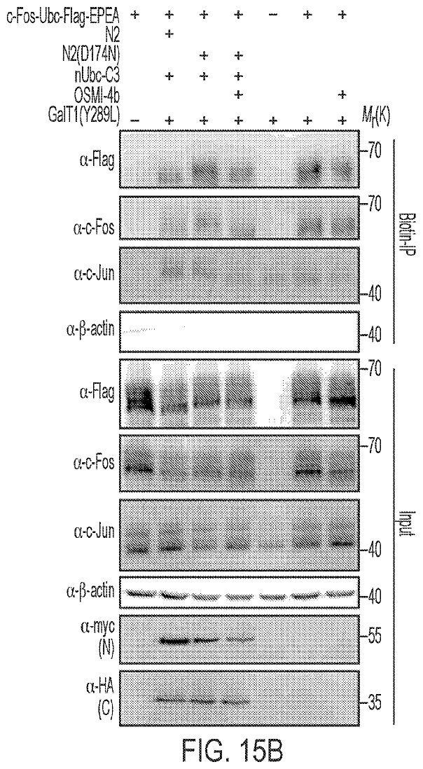

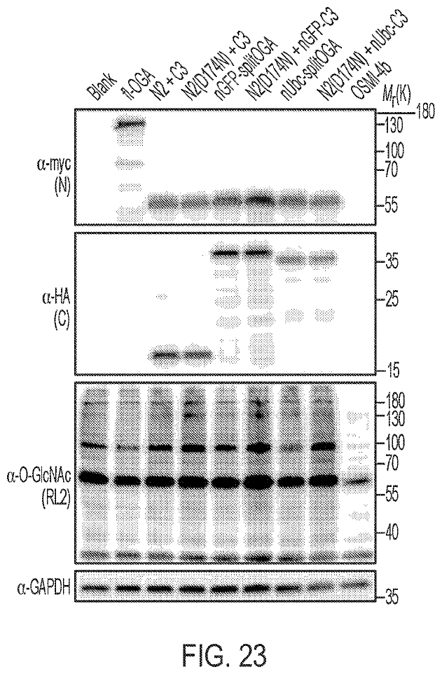

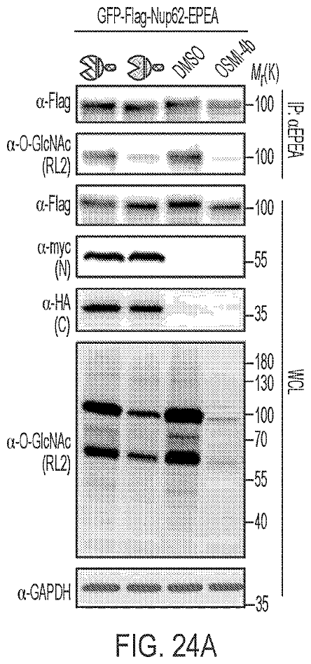

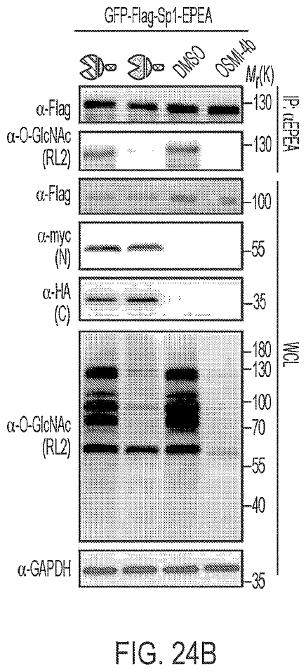

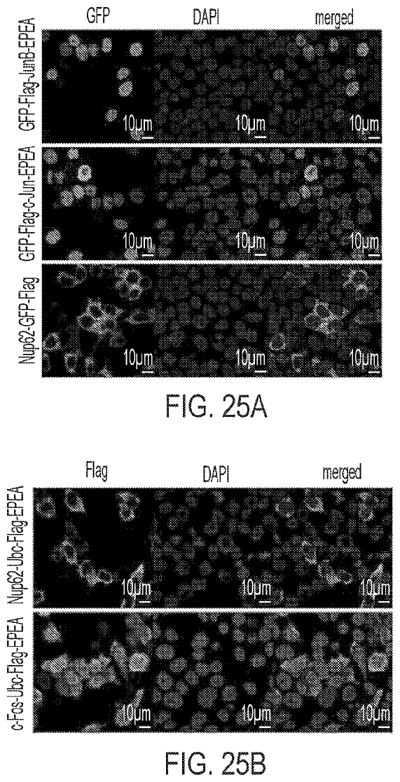

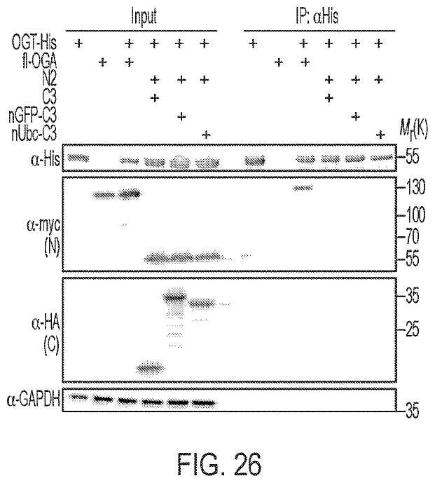

c-Jun forms a heterodimer with c-Fos as part of the AP-1 transcription factor complex. The deglycosylation process conducted by nUbc-split OGA on c-Fos-Ubc-Flag-EPEA was compared with the conventional strategy of treatment with the OGT inhibitor OSMI-4b (Martin, S. E. S. et al. Structure-Based Evolution of Low Nanomolar O-GlcNAc Transferase Inhibitors. J. Am. Chem. Soc. 2018, 140, 13542-13545) to explore a possible connection between O-GlcNAc and c-Fos on transcriptional activity by comparison of target protein deglycosylation to chemical inhibition of O-GlcNAcylation. c-Fos under different treatments was enriched and analyzed by immunoblotting. As shown in FIGS. 5C and 23, c-Fos-Ubc-Flag-EPEA was deglycosylated by both nUbc-split OGA and OGT inhibition. However, OGT inhibition by OSMI-4b induced a dramatic reduction of global O-GlcNAcylation level. By contrast, the O-GlcNAc modification on endogenous c-Jun was reduced upon OGT inhibition by OSMI-4b, but remained unperturbed under the treatment of nUbc-splitOGA (FIG. 15). These results indicate that nUbc-split OGA can selectively remove O-GlcNAc from target protein without interfering O-GlcNAcylation globally. It was next investigated whether removing O-GlcNAc on c-Fos would alter its transcriptional activity. An AP-1 responsive luciferase reporter (Kim, S., Maynard, J. C., Strickland, A., Burlingame, A. L. & Milbrandt, J. Schwann cell O-GlcNAcylation promotes peripheral nerve remyelination via attenuation of the AP-1 transcription factor JUN. Proc Natl Acad Sci USA 2018, 115, 8019-8024) and c-Fos-Ubc-Flag-EPEA were co-transfected in HEK293T cells. Upon OGT inhibition with OSMI-4b, the transcriptional activity of c-Fos-Ubc-Flag-EPEA was significantly upregulated as shown by the luciferase reporter assay (FIG. 5D). AP-1 transcriptional activity remained largely unperturbed on selective c-Fos deglycosylation using nUbc-splitOGA in comparison to the inactive mutant, indicating that direct deglycosylation of c-Fos is not promoting AP-1 activity (FIG. 5E). Addition of OSMI-4b again recovered AP-1 transcriptional activity in the presence of the inactive nUbc-splitOGA. Therefore, based on the insight garnered by the target protein deglycosylation approach, the enhanced transcriptional activity induced by OSMI-4b may be promoted by removal of O-GlcNAc from other proteins and is not directly linked to removal of O-GlcNAc on c-Fos. For example, AP-1 is composed of members from several protein families (Hess, J., et al. AP-1 subunits: quarrel and harmony among siblings. J. Cell Sci. 2004, 117, 5965-73), of which several are O-GlcNAc modified, and other members of transcription machinery are O-GlcNAc modified as well, such as RNA polymerase 1137 and TATA-binding protein (Hardiville, S. et al. TATA-box binding protein O-GlcNAcylation at T114 regulates formation of the B-TFIID complex and is critical for metabolic gene regulation. Mol. Cell 2020, 77, 1143-1152 e7). Taken together, the nanobody-fused split OGA can readily translate effects observed on the global O-GlcNAc proteome back to the desired target protein in cells and will find particular use in the study of target proteins bearing multiple or only partially characterized glycosites.

### Sequences

TABLE 1
Sequences

| SEQ | | |
|---|---|---|
| ID | | |
| NO: | Description | Sequence |
| 1 | ORF_myc- | MASMQKLISEEDLLMAMEARIRSTVQKESQ |
| | hOGA | ATLEERESELSSNPAASAGASLEPPAAPAP |
| | | GEDNPAGAGGAAVAGAAGGARRFLCGVVEG |
| | | FYGRPWVMEQRKELFRRLQKWELNTYLYAP |
| | | KDDYKHRMFWREMYSVEEAEQLMTLISAAR |
| | | EYEIEFIYAISPGLDITFSNPKEVSTLKRK |
| | | LDQVSQFGCRSFALLFDDIDHNMCAADKEV |
| | | FSSFAHAQVSITNEIYQYLGEPETFLFCPT |
| | | EYCGTFCYPNVSQSPYLRTVGEKLLPGIEV |
| | | LWTGPKVVSKEIPVESIEEVSKIIKRAPVI |
| | | WDNIHANDYDQKRLFLGPYKGRSTELIPRL |
| | | KGVLTNPNCEFEANYVAIHTLATWYKSNMN |
| | | GVRKDVVMTDSEDSTVSIQIKLENEGSDED |
| | | IETDVLYSPQMALKLALTEWLQEFGVPHQY |
| | | SSRQVAHSGAKASVVDGTPLVAAPSLNATT |
| | | VVTTVYQEPIMSQGAALSGEPTTLTKEEEK |
| | | KQPDEEPMDMVVEKQEETDHKNDNQILSEI |
| | | VEAKMAEELKPMDTDKESIAESKSPEMSMQ |
| | | EDCISDIAPMQTDEQTNKEQFVPGPNEKPL |
| | | YTAEPVTLEDLQLLADLFYLPYEHGPKGAQ |
| | | MLREFQWLRANSSVVSVNCKGKDSEKIEEW |
| | | RSRAAKFEEMCGLVMGMFTRLSNCANRTIL |
| | | YDMYSYVWDIKSIMSMVKSFVQWLGCRSHS |
| | | SAQFLIGDQEPWAFRGGLAGEFQRLLPIDG |
| | | ANDLFFQPPPLTPTSKVYTIRPYFPKDEAS |
| | | VYKICREMYDDGVGLPFQSQPDLIGDKLVG |
| | | GLLSLSLDYCFVLEDEDGICGYALGTVDVT |
| | | PFIKKCKISWIPFMQEKYTKPNGDKELSEA |
| | | EKIMLSFHEEQEVLPETFLANFPSLIKMDI |
| | | HKKVTDPSVAKSMMACLLSSLKANGSRGAF |
| | | CEVRPDDKRILEFYSKLGCFEIAKMEGFPK |
| | | DVVILGRSL* |
| 2 | ORF_myc- | ATGGCATCAATGCAGAAGCTGATCTCAGAG |
| | hOGA | GAGGACCTGCTTATGGCCATGGAGGCCCGA |
| | | ATTCGGTCGACCGTGCAGAAGGAGAGTCAA |
| | | GCGACGTTGGAGGAGCGGGAGAGCGAGCTC |
| | | AGCTCCAACCCTGCCGCCTCTGCGGGGGCA |
| | | TCGCTGGAGCCGCCGGCAGCTCCGGCACCC |
| | | GGAGAAGACAACCCCGCCGGGGCTGGGGGA |
| | | GCGGCGGTGGCCGGGGCTGCAGGAGGGGCT |
| | | CGGCGGTTCCTCTGCGGTGTGGTGGAAGGA |
| | | TTTTATGGAAGACCTTGGGTTATGGAACAG |
| | | AGAAAAGAACTCTTTAGAAGGCTCCAGAAA |
| | | TGGGAATTAAATACATACTTGTATGCCCCA |
| | | AAAGATGACTACAAACATAGGATGTTTTGG |
| | | CGAGAGATGTATTCAGTGGAGGAAGCTGAG |
| | | CAACTTATGACTCTCATCTCTGCTGCACGA |
| | | GAATATGAGATAGAGTTCATCTATGCGATC |
| | | TCACCTGGATTGGATATCACTTTTTCTAAC |
| | | CCCAAGGAAGTATCCACATTGAAACGTAAA |
| | | TTGGACCAGGTTTCTCAGTTTGGGTGCAGA |
| | | TCATTTGCTTTGCTTTTTGATGATATAGAC |
| | | CATAATATGTGTGCAGCAGACAAAGAGGTA |
| | | TTCAGTTCTTTTGCTCATGCCCAAGTCTCC |
| | | ATCACAAATGAAATCTATCAGTACCTAGGA |
| | | GAGCCAGAAACTTTCCTCTTCTGTCCCACA |
| | | GAATACTGTGGCACTTTCTGTTATCCAAAT |
| | | GTGTCTCAGTCTCCATATTTAAGGACTGTG |
| | | GGTGAAAAGCTTCTACCTGGAATTGAAGTG |
| | | CTTTGGACAGGTCCCAAAGTTGTTTCTAAA |
| | | GAAATTCCAGTAGAGTCCATCGAAGAGGTT |
| | | TCTAAGATTATTAAGAGAGCTCCAGTAATC |
| | | TGGGATAACATTCATGCTAATGATTATGAT |
| | | CAGAAGAGACTGTTTCTGGGCCCGTACAAA |
| | | GGAAGATCCACAGAACTCATCCCACGGTTA |
| | | AAAGGAGTCCTCACTAATCCAAATTGTGAA |
| | | TTTGAAGCCAACTACGTTGCTATCCACACC |
| | | CTTGCCACCTGGTACAAATCAAACATGAAT |
| | | GGAGTGAGAAAAGATGTAGTGATGACTGAC |
| | | AGTGAAGATAGTACTGTGTCCATCCAGATA |
| | | AAATTAGAAAATGAAGGCAGTGATGAAGAT |
| | | ATTGAAACTGATGTACTCTATAGTCCACAG |
| | | ATGGCTCTAAAGCTAGCATTAACAGAATGG |
| | | TTGCAAGAGTTTGGTGTGCCTCATCAATAC |
| | | AGCAGTAGGCAAGTTGCACACAGTGGAGCT |
| | | AAAGCAAGTGTAGTTGATGGGACTCCTTTA |
| | | GTTGCAGCACCCTCTTTAAATGCCACAACC |
| | | GTAGTAACAACAGTTTATCAGGAGCCCATT |
| | | ATGAGCCAGGGAGCAGCCTTGAGTGGTGAG |
| | | CCTACTACTCTGACCAAGGAAGAAGAAAAG |
| | | AAACAGCCTGATGAAGAACCCATGGACATG |
| | | GTGGTGGAAAAACAAGAAGAAACGGACCAC |
| | | AAGAATGACAATCAAATACTGAGTGAAATT |
| | | GTTGAAGCGAAAATGGCAGAGGAATTGAAA |
| | | CCAATGGACACTGATAAAGAGAGCATAGCT |
| | | GAATCAAAATCCCCAGAGATGTCCATGCAA |
| | | GAAGATTGTATTAGTGACATTGCCCCCATG |
| | | CAAACTGATGAACAGACAAACAAGGAGCAG |
| | | TTTGTGCCAGGTCCAAATGAAAAGCCTTTG |
| | | TACACTGCGGAACCAGTGACCCTGGAGGAT |
| | | TTGCAGTTACTTGCTGATCTATTCTACCTT |
| | | CCTTACGAGCATGGACCCAAAGGAGCACAG |
| | | ATGTTACGGGAATTTCAATGGCTTCGAGCA |
| | | AATAGTAGTGTTGTCAGTGTCAATTGCAAA |
| | | GGAAAAGACTCTGAAAAAATTGAAGAATGG |
| | | CGGTCACGAGCAGCCAAGTTTGAAGAGATG |
| | | TGTGGACTAGTGATGGGAATGTTCACTCGG |
| | | CTCTCCAATTGTGCCAACAGGACAATTCTT |
| | | TATGACATGTACTCCTATGTTTGGGATATC |
| | | AAGAGTATAATGTCTATGGTGAAGTCTTTT |
| | | GTACAGTGGTTAGGGTGTCGTAGTCATTCT |
| | | TCAGCACAATTCTTAATTGGAGACCAAGAA |
| | | CCCTGGGCCTTTAGAGGTGGTCTAGCAGGA |
| | | GAGTTCCAGCGTTTGCTGCCAATTGATGGG |
| | | GCAAATGATCTCTTTTTTCAGCCACCTCCA |
| | | CTGACTCCTACCTCCAAAGTTTATACTATC |
| | | AGACCTTATTTTCCTAAGGATGAGGCATCC |
| | | GTGTACAAGATTTGCAGAGAAATGTATGAC |
| | | GATGGAGTGGGTTTACCCTTTCAAAGTCAG |
| | | CCTGATCTTATTGGAGACAAGTTAGTAGGA |
| | | GGGCTGCTTTCCCTCAGCCTGGATTACTGC |
| | | TTTGTCCTAGAAGATGAAGATGGCATATGT |
| | | GGTTATGCCTTGGGCACTGTAGATGTGACC |
| | | CCCTTTATTAAAAAATGTAAAATTTCCTGG |
| | | ATCCCCTTCATGCAGGAGAAGTATACCAAG |
| | | CCAAATGGTGACAAGGAACTCTCTGAGGCT |
| | | GAGAAAATAATGTTGAGTTTCCATGAAGAA |
| | | CAGGAAGTACTGCCAGAAACTTTCCTTGCT |
| | | AATTTCCCTTCTCTGATAAAGATGGACATT |
| | | CACAAAAAAGTAACTGACCCAAGTGTGGCC |
| | | AAAAGCATGATGGCTTGCCTCCTGTCTTCA |
| | | CTGAAGGCTAATGGCTCCCGGGGAGCTTTC |
| | | TGTGAAGTGAGACCAGATGATAAAAGAATT |
| | | CTGGAATTTTACAGCAAGTTAGGATGTTTT |
| | | GAAATTGCAAAAATGGAAGGATTTCCAAAG |
| | | GATGTGGTTATACTTGGTCGGAGCCTGTAA |
| 3 | ORF_myc- | ATGGAGCAGAAGCTGATCAGCGAGGAGGAC |
| | OGA | CTGGCGATCGCAATGGTGCAGAAGGAGAGT |
| | (1-400) | CAAGCGACGTTGGAGGAGCGGGAGAGCGAG |
| | | CTCAGCTCCAACCCTGCCGCCTCTGCGGGG |
| | | GCATCGCTGGAGCCGCCGGCAGCTCCGGCA |
| | | CCCGGAGAAGACAACCCCGCCGGGGCTGGG |
| | | GGAGCGGCGGTGGCCGGGGCTGCAGGAGGG |
| | | GCTCGGCGGTTCCTCTGCGGTGTGGTGGAA |
| | | GGATTTTATGGAAGACCTTGGGTTATGGAA |
| | | CAGAGAAAAGAACTCTTTAGAAGGCTCCAG |
| | | AAATGGGAATTAAATACATACTTGTATGCC |
| | | CCAAAAGATGACTACAAACATAGGATGTTT |
| | | TGGCGAGAGATGTATTCAGTGGAGGAAGCT |
| | | GAGCAACTTATGACTCTCATCTCTGCTGCA |
| | | CGAGAATATGAGATAGAGTTCATCTATGCG |
| | | ATCTCACCTGGATTGGATATCACTTTTTCT |
| | | AACCCCAAGGAAGTATCCACATTGAAACGT |
| | | AAATTGGACCAGGTTTCTCAGTTTGGGTGC |
| | | AGATCATTTGCTTTGCTTTTTGATGATATA |
| | | GACCATAATATGTGTGCAGCAGACAAAGAG |
| | | GTATTCAGTTCTTTTGCTCATGCCCAAGTC |
| | | TCCATCACAAATGAAATCTATCAGTACCTA |
| | | GGAGAGCCAGAAACTTTCCTCTTCTGTCCC |
| | | ACAGAATACTGTGGCACTTTCTGTTATCCA |
| | | AATGTGTCTCAGTCTCCATATTTAAGGACT |
| | | GTGGGTGAAAAGCTTCTACCTGGAATTGAA |
| | | GTGCTTTGGACAGGTCCCAAAGTTGTTTCT |
| | | AAAGAAATTCCAGTAGAGTCCATCGAAGAG |
| | | GTTTCTAAGATTATTAAGAGAGCTCCAGTA |
| | | ATCTGGGATAACATTCATGCTAATGATTAT |
| | | GATCAGAAGAGACTGTTTCTGGGCCCGTAC |
| | | AAAGGAAGATCCACAGAACTCATCCCACGG |
| | | TTAAAAGGAGTCCTCACTAATCCAAATTGT |
| | | GAATTTGAAGCCAACTACGTTGCTATCCAC |
| | | ACCCTTGCCACCTGGTACAAATCAAACATG |
| | | AATGGAGTGAGAAAAGATGTAGTGATGACT |
| | | GACAGTGAAGATAGTACTGTGTCCATCCAG |
| | | ATAAAATTAGAAAATGAAGGCAGTGATGAA |
| | | GATATTGAAACTGATGTACTCTATAGTCCA |
| | | CAGATGGCTCTAAAGCTAGCATTAACAGAA |
| | | TGGTTGCAAGAGTTTGGTGTGCCTCATCAA |
| | | TACAGCAGTAGGTAA |
| 4 | ORF_myc- | MEQKLISEEDLAIAMVQKESQATLEERESE |
| | OGA | LSSNPAASAGASLEPPAAPAPGEDNPAGAG |
| | (1-400) | GAAVAGAAGGARRFLCGVVEGFYGRPWVME |
| | | QRKELFRRLQKWELNTYLYAPKDDYKHRMF |
| | | WREMYSVEEAEQLMTLISAAREYEIEFIYA |
| | | ISPGLDITFSNPKEVSTLKRKLDQVSQFGC |
| | | RSFALLFDDIDHNMCAADKEVFSSFAHAQV |
| | | SITNEIYQYLGEPETFLFCPTEYCGTFCYP |
| | | NVSQSPYLRTVGEKLLPGIEVLWTGPKVVS |
| | | KEIPVESIEEVSKIIKRAPVIWDNIHANDY |
| | | DQKRLFLGPYKGRSTELIPRLKGVLTNPNC |
| | | EFEANYVAIHTLATWYKSNMNGVRKDVVMT |
| | | DSEDSTVSIQIKLENEGSDEDIETDVLYSP |
| | | QMALKLALTEWLQEFGVPHQYSSR* |
| 5 | ORF_myc- | ATGGAGCAGAAGCTGATCAGCGAGGAGGAC |
| | OGA | CTGGCGATCGCAATGGTGCAGAAGGAGAGT |
| | (1-400) | CAAGCGACGTTGGAGGAGCGGGAGAGCGAG |
| | D174N | CTCAGCTCCAACCCTGCCGCCTCTGCGGGG |
| | | GCATCGCTGGAGCCGCCGGCAGCTCCGGCA |
| | | CCCGGAGAAGACAACCCCGCCGGGGCTGGG |
| | | GGAGCGGCGGTGGCCGGGGCTGCAGGAGGG |
| | | GCTCGGCGGTTCCTCTGCGGTGTGGTGGAA |
| | | GGATTTTATGGAAGACCTTGGGTTATGGAA |
| | | CAGAGAAAAGAACTCTTTAGAAGGCTCCAG |
| | | AAATGGGAATTAAATACATACTTGTATGCC |
| | | CCAAAAGATGACTACAAACATAGGATGTTT |
| | | TGGCGAGAGATGTATTCAGTGGAGGAAGCT |
| | | GAGCAACTTATGACTCTCATCTCTGCTGCA |
| | | CGAGAATATGAGATAGAGTTCATCTATGCG |
| | | ATCTCACCTGGATTGGATATCACTTTTTCT |
| | | AACCCCAAGGAAGTATCCACATTGAAACGT |
| | | AAATTGGACCAGGTTTCTCAGTTTGGGTGC |
| | | AGATCATTTGCTTTGCTTTTTAATGATATA |
| | | GACCATAATATGTGTGCAGCAGACAAAGAG |
| | | GTATTCAGTTCTTTTGCTCATGCCCAAGTC |
| | | TCCATCACAAATGAAATCTATCAGTACCTA |
| | | GGAGAGCCAGAAACTTTCCTCTTCTGTCCC |
| | | ACAGAATACTGTGGCACTTTCTGTTATCCA |
| | | AATGTGTCTCAGTCTCCATATTTAAGGACT |
| | | GTGGGTGAAAAGCTTCTACCTGGAATTGAA |
| | | GTGCTTTGGACAGGTCCCAAAGTTGTTTCT |
| | | AAAGAAATTCCAGTAGAGTCCATCGAAGAG |
| | | GTTTCTAAGATTATTAAGAGAGCTCCAGTA |
| | | ATCTGGGATAACATTCATGCTAATGATTAT |
| | | GATCAGAAGAGACTGTTTCTGGGCCCGTAC |
| | | AAAGGAAGATCCACAGAACTCATCCCACGG |
| | | TTAAAAGGAGTCCTCACTAATCCAAATTGT |
| | | GAATTTGAAGCCAACTACGTTGCTATCCAC |
| | | ACCCTTGCCACCTGGTACAAATCAAACATG |
| | | AATGGAGTGAGAAAAGATGTAGTGATGACT |
| | | GACAGTGAAGATAGTACTGTGTCCATCCAG |
| | | ATAAAATTAGAAAATGAAGGCAGTGATGAA |
| | | GATATTGAAACTGATGTACTCTATAGTCCA |
| | | CAGATGGCTCTAAAGCTAGCATTAACAGAA |
| | | TGGTTGCAAGAGTTTGGTGTGCCTCATCAA |
| | | TACAGCAGTAGGTAA |
| 6 | ORF_myc- | MEQKLISEEDLAIAMVQKESQATLEERESE |
| | OGA | LSSNPAASAGASLEPPAAPAPGEDNPAGAG |
| | (1-400) | GAAVAGAAGGARRFLCGVVEGFYGRPWVME |
| | D174N | QRKELFRRLQKWELNTYLYAPKDDYKHRMF |
| | | WREMYSVEEAEQLMTLISAAREYEIEFIYA |
| | | ISPGLDITFSNPKEVSTLKRKLDQVSQFGC |
| | | RSFALLFNDIDHNMCAADKEVFSSFAHAQV |
| | | SITNEIYQYLGEPETFLFCPTEYCGTFCYP |
| | | NVSQSPYLRTVGEKLLPGIEVLWTGPKVVS |
| | | KEIPVESIEEVSKIIKRAPVIWDNIHANDY |
| | | DQKRLFLGPYKGRSTELIPRLKGVLTNPNC |
| | | EFEANYVAIHTLATWYKSNMNGVRKDVVMT |
| | | DSEDSTVSIQIKLENEGSDEDIETDVLYSP |
| | | QMALKLALTEWLQEFGVPHQYSSR* |
| 7 | ORF_HA- | ATGGCATACCCATACGATGTTCCAGATTAC |
| | OGA | GCTGCGATCGCAGAAAAGCCTTTGTACACT |
| | (544-706) | GCGGAACCAGTGACCCTGGAGGATTTGCAG |
| | | TTACTTGCTGATCTATTCTACCTTCCTTAC |
| | | GAGCATGGACCCAAAGGAGCACAGATGTTA |
| | | CGGGAATTTCAATGGCTTCGAGCAAATAGT |
| | | AGTGTTGTCAGTGTCAATTGCAAAGGAAAA |
| | | GACTCTGAAAAAATTGAAGAATGGCGGTCA |
| | | CGAGCAGCCAAGTTTGAAGAGATGTGTGGA |
| | | CTAGTGATGGGAATGTTCACTCGGCTCTCC |
| | | AATTGTGCCAACAGGACAATTCTTTATGAC |
| | | ATGTACTCCTATGTTTGGGATATCAAGAGT |
| | | ATAATGTCTATGGTGAAGTCTTTTGTACAG |
| | | TGGTTAGGGTGTCGTAGTCATTCTTCAGCA |
| | | CAATTCTTAATTGGAGACCAAGAACCCTGG |
| | | GCCTTTAGAGGTGGTCTAGCAGGAGAGTTC |
| | | CAGCGTTTGCTGCCAATTGATGGGGCAAAT |
| | | GATCTCTTTTTTCAGCCACCTTAA |
| 8 | ORF_HA- | MAYPYDVPDYAAIAEKPLYTAEPVTLEDLQ |
| | OGA | LLADLFYLPYEHGPKGAQMLREFQWLRANS |
| | (544-706) | SVVSVNCKGKDSEKIEEWRSRAAKFEEMCG |
| | | LVMGMFTRLSNCANRTILYDMYSYVWDIKS |
| | | IMSMVKSFVQWLGCRSHSSAQFLIGDQEPW |
| | | AFRGGLAGEFQRLLPIDGANDLFFQPP* |
| 9 | ORF_HA- | ATGGCATACCCATACGATGTTCCAGATTAC |
| | nGFP- | GCTGCGATCGCACAGGTGCAGCTGGTGGAG |
| | (EAAAK)4- | TCTGGAGGAGCTCTGGTGCAGCCTGGAGGA |
| | OGA | AGCCTGCGCCTGAGCTGTGCAGCTAGCGGA |
| | (544-706) | TTTCCTGTGAACCGCTACAGCATGCGCTGG |
| | | TACCGCCAGGCTCCTGGTAAAGAGCGCGAG |
| | | TGGGTGGCTGGAATGAGCAGCGCTGGAGAT |
| | | CGCAGCAGCTACGAGGACAGCGTGAAAGGA |
| | | CGCTTTACAATCAGCCGCGATGATGCTCGC |
| | | AACACAGTGTACCTGCAGATGAACTCTCTG |
| | | AAACCTGAGGACACTGCTGTGTACTACTGT |
| | | AACGTGAACGTGGGTTTCGAGTACTGGGGA |
| | | CAGGGAACACAGGTGACAGTGAGCTCTGGC |
| | | GCGCCAGAGGCAGCTGCAAAGGAGGCAGCT |
| | | GCAAAGGAGGCAGCTGCAAAGGAGGCAGCT |
| | | GCAAAGTTAATTAAGGAAAAGCCTTTGTAC |
| | | ACTGCGGAACCAGTGACCCTGGAGGATTTG |
| | | CAGTTACTTGCTGATCTATTCTACCTTCCT |
| | | TACGAGCATGGACCCAAAGGAGCACAGATG |
| | | TTACGGGAATTTCAATGGCTTCGAGCAAAT |
| | | AGTAGTGTTGTCAGTGTCAATTGCAAAGGA |
| | | AAAGACTCTAAAAAAATTGAAGAATGGCGG |
| | | TCACGAGCAGCCAAGTTTGAAGAGATGTGT |
| | | GGACTAGTGATGGGAATGTTCACTCGGCTC |
| | | TCCAATTGTGCCAACAGGACAATTCTTTAT |
| | | GACATGTACTCCTATGTTTGGGATATCAAG |
| | | AGTATAATGTCTATGGTGAAGTCTTTTGTA |
| | | CAGTGGTTAGGGTGTCGTAGTCATTCTTCA |
| | | GCACAATTCTTAATTGGAGACCAAGAACCC |
| | | TGGGCCTTTAGAGGTGGTCTAGCAGGAGAG |
| | | TTCCAGCGTTTGCTGCCAATTGATGGGGCA |
| | | AATGATCTCTTTTTTCAGCCACCTTAA |
| 10 | ORF_HA- | MAYPYDVPDYAAIAQVQLVESGGALVQPGG |
| | nGFP- | SLRLSCAASGFPVNRYSMRWYRQAPGKERE |
| | (EAAAK)4- | WVAGMSSAGDRSSYEDSVKGRFTISRDDAR |
| | OGA | NTVYLQMNSLKPEDTAVYYCNVNVGFEYWG |
| | (544-706) | QGTQVTVSSGAPEAAAKEAAAKEAAAKEAA |
| | | AKLIKEKPLYTAEPVTLEDLQLLADLFYLP |
| | | YEHGPKGAQMLREFQWLRANSSVVSVNCKG |
| | | KDSKKIEEWRSRAAKFEEMCGLVMGMFTRL |
| | | SNCANRTILYDMYSYVWDIKSIMSMVKSFV |
| | | QWLGCRSHSSAQFLIGDQEPWAFRGGLAGE |
| | | FQRLLPIDGANDLFFQPP* |

TABLE 2
List of plasmids

| No. | Plasmid name | Abbreviated Names | Details |
|---|---|---|---|
| 1 | pCMV-myc-OGA | fl-OGA | Full length OGA with a |
| | | | myc tag |
| 2 | pcDNA3.1-myc-OGA(cat) | myc-OGA(cat)/N3 | |
| 3 | pcDNA3.1-myc-OGA(ΔHAT) | | |
| 4 | pcDNA3.1-myc-OGA(GS-ΔHAT) | | |
| 5 | pcDNA3.1-HA-OGA | | Full length OGA with a |
| | | | HA tag |
| 6 | pcDNA3.1-HA-nGFP-(EAAAK)4- | | A linker with four |
| | OGA | | (EAAAK) repeats is |
| 7 | pcDNA3.1-HA-nGFP-(EAAAK)4- | | used between nGFP |
| | OGA(cat) | | and indicated OGAs. |
| 8 | pcDNA3.1-HA-nGFP-(EAAAK)4- | | |
| | OGA(ΔHAT) | | |
| 9 | pcDNA3.1-HA-nGFP-(EAAAK)4- | | |
| | OGA(GS-ΔHAT) | | |
| 10 | pcDNA3.1-myc-OGA(1-413) | N1 | myc-tag for N |
| 11 | pcDNA3.1-myc-OGA(1-400) | N2 | fragments and HA-tag |
| 12 | pcDNA3.1-myc-OGA(1-400)D174N | N2(D174N) | for C fragments |
| 13 | pcDNA3.1-HA-OGA(414-916) | C1 | |
| 14 | pcDNA3.1-HA-OGA(414-706) | C2 | |
| 15 | pcDNA3.1-HA-OGA(544-706) | C3 | |
| 16 | pcDNA3.1-HA-OGA(554-706) | C4 | |
| 17 | pcDNA3.1-myc-nGFP-OGA(cat) | nGFP-OGA(cat)/N3 | A linker with four |
| 18 | pcDNA3.1-myc-nGFP-(EAAAK)4- | nGFP-N2 | (EAAAK) repeats is |
| | OGA(1-400) | | used between nGFP |
| 19 | pcDNA3.1-myc-nGFP-(EAAAK)4- | nGFP-N2(D174N) | and indicated OGAs; |
| | OGA(1-400)D174N | | myc-tag for N |
| 20 | pcDNA3.1-myc-nGFP-(EAAAK)4- | nGFP-OGA(GS- | fragments and HA-tag |
| | OGA(GS-AHAT) | ΔHAT) | for C fragments |
| 21 | pcDNA3.1-HA-nGFP-(EAAAK)4- | nGFP-C3 | |
| | OGA(544-706) | | |
| 22 | pcDNA3.1-HA-nGFP-(EAAAK)4- | nGFP-C4 | |
| | OGA(554-706) | | |
| 23 | pcDNA3.1-Nup62-GFP-Flag | Nup62-GFP-Flag | |
| 24 | pcDNA3.1-GFP-Flag-Nup62-EPEA | GFP-Nup62 | GFP and Flag tag at N |
| 25 | pcDNA3.1-GFP-Flag-Sp1-EPEA | GFP-Sp1 | terminus; EPEA tag at |
| 26 | pcDNA3.1-GFP-Flag-JunB-EPEA | GFP-JunB | C terminus |
| 27 | pcDNA3.1-GFP-Flag-c-Jun-EPEA | GFP-c-Jun | |
| 28 | pcDNA3.1-HA-nUbc-(EAAAK)4- | nUbc-C3 | nUbc-fused C3 |
| | OGA(544-706) | | fragment |
| 29 | pcDNA3.1-HA-nEPEA-(EAAAK)4- | nEPEA-C3 | nEPEA-fused C3 |
| | OGA(544-706) | | fragment |
| 30 | pcDNA3.1-HA-nBC2-(EAAAK)4- | nBC2-C3 | nBC2-fused C3 |
| | OGA(544-706) | | fragment |
| 31 | pcDNA3.1-Nup62-Ubc-Flag-EPEA | Nup62-Ubc-EPEA | Ubc, Flag and EPEA |
| 32 | pcDNA3.1-c-Fos-Ubc-Flag-EPEA | c-Fos-Ubc | tag at C terminus |
| 33 | pcDNA3.1-BC2-Nup62-Flag-EPEA | BC2-Nup62-EPEA | BC2 tag at N terminus; |
| | | | Flag and EPEA at C |
| | | | terminus |
| 34 | 3xAP1pGL3 | | AP-1 responsive |
| | | | luciferase reporter |
| 35 | pCMV3-OGT-His | OGT-His | His tag at C terminus |

### EQUIVALENTS AND SCOPE, INCORPORATION BY REFERENCE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

1. A fusion protein comprising (i) a nanobody, and (ii) a split glycosyl hydrolase comprising more than one piece; wherein the nanobody is fused to at least one piece of the split glycosyl hydrolase.

2. The fusion protein of claim 1, wherein the split glycosyl hydrolase is a split O-GlcNAcase (OGA).

3. The fusion protein of claim 1, wherein the split glycosyl hydrolase comprises (i) a first piece comprising a catalytic domain, and (ii) a second piece comprising a stalk domain.

4. The fusion protein of claim 3, wherein the catalytic domain comprises a truncated catalytic domain.

5. The fusion protein of claim 3, wherein the stalk domain comprises a truncated stalk domain.

6. (canceled)

7. The fusion protein of claim 3, wherein the catalytic domain comprises amino acid residues 1-400 of SEQ ID NO: 1.

8. The fusion protein of claim 3, wherein the stalk domain comprises amino acid residues 544-706 of SEQ ID NO: 1.

9. (canceled)

10. The fusion protein of claim 1, wherein the nanobody is fused to the N terminus of a piece that comprises a stalk domain.

11. The fusion protein of claim 1, wherein the nanobody binds a cell surface protein.

12. The fusion protein of claim 1, wherein the nanobody binds a protein selected from the group of GFP, EPEA, and UBC6e.

13-14. (canceled)

15. The fusion protein of claim 1, wherein the nanobody and at least one piece of the split glycosyl hydrolase is fused via a peptide linker.

16. A split OGA enzyme comprising (i) a first piece comprising a truncated catalytic domain, and (ii) a second piece comprising a truncated stalk domain.

17. A pharmaceutical composition comprising the fusion protein of claim 1 pharmaceutically acceptable excipient.

18. A polynucleotide encoding the fusion protein of claim 1.

19. A vector comprising the polynucleotide of claim 18.

20. A cell comprising the fusion protein of claim 1.

21. A cell comprising the polynucleotide of claim 18.

22. A method of deglycosylating a protein, the method comprising:
contacting a target protein containing a sugar moiety with the fusion protein of claim 1, wherein the sugar moiety is removed from the target protein.

23-28. (canceled)

29. A method of treating a glycosylation-associated disease in a subject, the method comprising administering to the subject a therapeutically effective amount of the fusion protein of claim 1 to a patient in need thereof.

30-33. (canceled)

34. A kit comprising the fusion protein of claim 1.
